(19) United States
(12) Patent Application Publication
(10) Pub. No.: US 2015/0239918 A1
(43) Pub. Date: Aug. 27, 2015

(54) FUNCTIONALIZED CARBON MATRIX MATERIALS

(71) Applicants: Robert Lee Johnson, Ames, IA (US); Jason Anderson, Ames, IA (US); Klaus Schmidt-Rohr, Ames, IA (US); Brent Shanks, Ames, IA (US)

(72) Inventors: Robert Lee Johnson, Ames, IA (US); Jason Anderson, Ames, IA (US); Klaus Schmidt-Rohr, Ames, IA (US); Brent Shanks, Ames, IA (US)

(21) Appl. No.: 14/192,430

(22) Filed: Feb. 27, 2014

(51) Int. Cl. C07F 9/572; C07C 303/22; C07D 401/14; C07D 401/06; A61K 49/00; B01J 31/02; B01J 37/16; B01J 37/08; G21F 1/10; C07C 309/14; C07D 211/26

(52) U.S. Cl. CPC C07F 9/5722; C07C 309/14; C07C 303/22; C07D 401/14; C07D 401/06; C07D 211/26; B01J 31/0271; B01J 31/0244; B01J 31/0267; B01J 37/16; B01J 37/084; G21F 1/10; A61K 49/0002; B01J 2531/004; B01J 2231/49

(57) ABSTRACT

Various embodiments of the present invention provide a functionalized carbon matrix, compositions and composites including the same, and methods of making and using the same. In various embodiments, the present invention provides a functionalized carbon matrix having the structure CM-(NZ-L-FG)ₙ. At each occurrence, Z is independently selected from H and a bond to CM. The variable CM is a substituted or unsubstituted carbon matrix. At each occurrence, linker L is independently substituted or unsubstituted. At each occurrence, variable FG is independently selected from a functional group. The degree of substitution n is about 1 to about 1,000,000.

### STATEMENT OF GOVERNMENT SUPPORT

This invention was made with U.S. Government support under contracts EEC-0813570 and DBI 9413969 awarded by the National Science Foundation. The U.S. Government has certain rights in this invention.

### BACKGROUND OF THE INVENTION

Carbon materials containing acidic and/or basic heteroatom functional groups are promising for numerous applications, including heterogeneous catalysis, adsorbents, polymer electrolyte fuel cells, anodes in lithium ion batteries, and capacitors. Carbon materials with high nitrogen loading have been shown to be very effective as a support for highly active and distributed Pd nanoparticles. Moreover, carbon materials are better suited compared to metal oxides or silica for applications that require resistance to degradation in liquid phase, including acids, bases, and water under high temperature and pressures (hydrothermal conditions). Uniformly functionalized carbon materials are also good precursors for nanocomposites.

A few general approaches have been employed to produce functionalized carbon materials. These methods are limited in the types of functional groups that can be introduced and in the amounts of functional-group incorporation that can be achieved.

Consumption of sulfuric acid, in a variety of different industries, exceeds 15 million tons per year and can be used as an industrial economic indicator in the US. One of the major uses of H₂SO₄ is as a homogeneous acid catalyst. Such acid catalysts are important in a number of different reactions such as esterification, dehydration, and etherification. Typically the reactions proceed through a carbocation intermediate and transform the simple commodity chemicals into value-added products. By using acid catalysts, the chemical industry transforms a handful of chemicals into the thousands used in today's society. However, sulfuric acid is difficult to separate from the reaction and is generally treated prior to discharging to the environment. Therefore researchers are interested in discovering a heterogeneous catalyst that is similar in price and activity as sulfuric acid. Typical catalyst supports used in the petroleum industry include alumina and zeolites. However, as oil and non-renewable resources become expensive, interest has grown in using renewable resources, where typically carbohydrates like glucose are the primary feedstock. Unlike petroleum products, this feedstock cannot be chemically transformed in the gas phase since it degrades long before being volatilized. In turn, the catalyst supports used in the petroleum industry degrade under hot water conditions and therefore cannot be used with biorenewable processing. Available catalysts do not have the hydrothermal stability to withstand condensed-phase processing.

### SUMMARY OF THE INVENTION

In various embodiments, the present invention provides a functionalized carbon matrix having the structure:

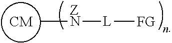

At each occurrence, Z is independently selected from H and a bond to CM. The variable CM is a substituted or unsubstituted carbon matrix. At each occurrence linker L is independently substituted or unsubstituted (C₁-C₄₀)hydrocarbyl interrupted by 0, 1, 2, or 3 atoms chosen from —S—, —O—, and —NR³—. At each occurrence functional group FG is independently chosen from —OR³, —OOR³, —OC(O)N(R³)₂, —CN, —CF₃, —OCF₃, —C(O), methylenedioxy, ethylenedioxy, —N(R³)₂, —SR³, —SOR³, —SO₂N(R³)₂, —C(O)R³, —C(O)C(O)R³, —C(O)CH₂C(O)R³, —C(S)R³, —C(O)OR³, —OC(O)R³, —C(O)N(R³)₂, —OC(O)N(R³)₂, —C(S)N(R³)₂, —N(R³)C(O)R³, —N(R³)N(R³)₂, —N(R³)N(R³)C(O)R³, —N(R³)N(R³)C(O)OR³, —N(R³)N(R³)CON(R³)₂, —N(R³)SO₂R³, —N(R³)SO₂N(R³)₂, —N(R³)C(O)OR³, —N(R³)C(O)R³, —N(R³)C(S)R³, —N(R³)C(O)N(R³)₂, —N(R³)C(S)N(R³)₂, —N(COR³)COR³, —N(OR³)R³, —C(═NH)N(R³)₂, —C(O)N(OR³)R³, —C(═NOR³)R³, —S(O)(OR¹)₃, —S(O)(OR¹)₂R², —S(O)(OR¹)R²₂, —S(O)R²₂, —OS(O)(OR¹)₃, —OS(O)(OR¹)₂R², —OS(O)(OR¹)R²₂, —OS(O)R²₂, —S(O)₂OR¹, —S(O)₂R², —OS(O)₂OR¹, —OS(O)₂R², —P(O)(OR¹)₂, —P(O)(OR¹)R², —P(O)R²₂, —OP(O)(OR¹)₂, —OP(O)(OR¹)R², —OP(O)R²₂, and a substituted or unsubstituted nitrogen-containing (C₁-C₂₀)heterocycle. At each occurrence R¹ is independently chosen from —H, a counterion, and substituted or unsubstituted (C₁-C₂₀)hydrocarbyl. At each occurrence R² is independently chosen from substituted or unsubstituted (C₁-C₂₀)hydrocarbyl. At each occurrence R³ is independently chosen from —H and substituted or unsubstituted (C₁-C₂₀)hydrocarbyl. The degree of substitution n is about 1 to about 1,000,000.

In various embodiments the present invention provides a functionalized carbon matrix having the structure:

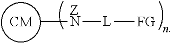

At each occurrence, Z is independently selected from H and a bond to CM. The variable CM is a substituted or unsubstituted carbon matrix. At each occurrence L is chosen from ethylene, butylene, methylene, and phenylene. At each occurrence FG is chosen from S(O)₂OH, P(O)(OH)₂, pyridin-4-yl, and piperidin-4-yl. The degree of substitution n is about 1 to about 1,000,000.

In various embodiments, the present invention provides a method of making a functionalized carbon matrix. The method includes obtaining or providing a composition comprising a reducing sugar and a functionalized amine having the structure H₂N-L-FG. At each occurrence linker L is independently substituted or unsubstituted (C₁-C₄₀)hydrocarbyl interrupted by 0, 1, 2, or 3 atoms chosen from —S—, —O—, and —NR³—. At each occurrence functional group FG is independently chosen from —OR³, —OOR³, —OC(O)N(R³)₂, —CN, —CF₃, —OCF₃, —C(O), methylenedioxy, ethylenedioxy, —N(R³)₂, —SR³, —SOR³, —SO₂N(R³)₂, —C(O)R³, —C(O)C(O)R³, —C(O)CH₂C(O)R³, —C(S)R³, —C(O)OR³, —OC(O)R³, —C(O)N(R³)₂, —OC(O)N(R³)₂, —C(S)N(R³)₂, —N(R³)C(O)R³, —N(R³)N(R³)₂, —N(R³)N(R³)C(O)R³, —N(R³)N(R³)C(O)OR³, —N(R³)N(R³)CON(R³)₂, —N(R³)SO₂R³, —N(R³)SO₂N(R³)₂, —N(R³)C(O)OR³, —N(R³)C(O)R³, —N(R³)C(S)R³, —N(R³)C(O)N(R³)₂, —N(R³)C(S)N(R³)₂, —N(COR³)COR³, —N(OR³)R³, —C(═NH)N(R³)₂, —C(O)N(OR³)R³, —C(═NOR³)R³, —S(O)(OR¹)₃, —S(O)(OR¹)₂R², —S(O)(OR¹)R²₂, —S(O)R²₂, —OS(O)(OR¹)₃, —OS(O)(OR¹)₂R², —OS(O)(OR¹)₂R², —OS(O)R²₂, —S(O)₂OR¹, —S(O)₂R², —OS(O)₂OR¹, —OS(O)₂R², —P(O)(OR¹)₂, —P(O)(OR¹)R², —P(O)R²₂, —OP(O)(OR¹)₂, —OP(O)(OR¹)R², —OP(O)R²₂, and a substituted or unsubstituted nitrogen-containing (C₁-C₂₀)heterocycle. At each occurrence R¹ is independently chosen from —H, a counterion, and substituted or unsubstituted (C₁-C₂₀)hydrocarbyl. At each occurrence R² is independently chosen from substituted or unsubstituted (C₁-C₂₀)hydrocarbyl. At each occurrence R³ is independently chosen from —H and substituted or unsubstituted (C₁-C₂₀)hydrocarbyl. The method also includes subjecting the composition to at least one of pyrolysis and hydrothermal carbonization, to provide a functionalized carbon matrix having the structure:

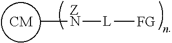

At each occurrence, Z is independently selected from H and a bond to CM. The variable CM is a substituted or unsubstituted carbon matrix and degree of substitution n is about 1 to about 1,000,000.

In various embodiments the present invention provides a method of making a functionalized carbon matrix. The method includes obtaining or providing a composition including glucose and a functionalized amine chosen from aminoethyl sulfonic acid, aminoethyl phosphonic acid, 4-(2-aminoethyl)pyridine and 4-aminomethylpiperidine. The method also includes subjecting the composition to at least one of pyrolysis and hydrothermal carbonization, to provide a functionalized carbon matrix having the structure:

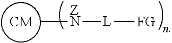

At each occurrence, Z is independently selected from H and a bond to CM. The variable CM is a substituted or unsubstituted carbon matrix. At each occurrence L is independently chosen from methylene, ethylene, butylene, and phenylene. At each occurrence FG is independently chosen from S(O)₂OH, P(O)(OH)₂, pyridin-4-yl, and piperidin-4-yl. At each occurrence degree of substitution n is about 1 to about 1,000,000.

In various embodiments, the present invention provides a method of making a nitrogen-rich carbon material. The method includes obtaining or providing a composition including a reducing sugar, and a functionalized amine having the structure H₂N-L-FG. At each occurrence linker L is independently substituted or unsubstituted (C₁-C₄₀)hydrocarbyl interrupted by 0, 1, 2, or 3 atoms chosen from —S—, —O—, and —NR³—. At each occurrence functional group FG is independently chosen from —OR³, —OOR³, —OC(O)N(R³)₂, —CN, —CF₃, —OCF₃, —C(O), methylenedioxy, ethylenedioxy, —N(R³)₂, —SR³, —SOR³, —SO₂N(R³)₂, —C(O)R³, —C(O)C(O)R³, —C(O)CH₂C(O)R³, —C(S)R³, —C(O)OR³, —OC(O)R³, —C(O)N(R³)₂, —OC(O)N(R³)₂, —C(S)N(R³)₂, —N(R³)C(O)R³, —N(R³)N(R³)₂, —N(R³)N(R³)C(O)R³, —N(R³)N(R³)C(O)OR³, —N(R³)N(R³)CON(R³)₂, —N(R³)SO₂R³, —N(R³)SO₂N(R³)₂, —N(R³)C(O)OR³, —N(R³)C(O)R³, —N(R³)C(S)R³, —N(R³)C(O)N(R³)₂, —N(R³)C(S)N(R³)₂, —N(COR³)COR³, —N(OR³)R³, —C(═NH)N(R³)₂, —C(O)N(OR³)R³, —C(═NOR³)R³, —S(O)(OR¹)₃, —S(O)(OR¹)₂R², —S(O)(OR¹)R²₂, —S(O)R²₂, —OS(O)(OR¹)₃, —OS(O)(OR¹)₂R², —OS(O)(OR¹)R²₂, —OS(O)R²₂, —S(O)₂OR¹, —S(O)₂R², —OS(O)₂OR¹, —OS(O)₂R², —P(O)(OR¹)₂, —P(O)(OR¹)R², —P(O)R²₂, —OP(O)(OR¹)₂, —OP(O)(OR¹)R², —OP(O)R²₂, and a substituted or unsubstituted nitrogen-containing (C₁-C₂₀)heterocycle. At each occurrence R¹ is independently chosen from —H, a counterion, and substituted or unsubstituted (C₁-C₂₀)hydrocarbyl. At each occurrence R² is independently chosen from substituted or unsubstituted (C₁-C₂₀)hydrocarbyl. At each occurrence R³ is independently chosen from —H and substituted or unsubstituted (C₁-C₂₀)hydrocarbyl. The method also includes subjecting the composition to pyrolysis comprising a temperature of about 500° C. to about 2000° C., to provide a nitrogen-rich carbon material.

Various embodiments of the present invention have certain advantages over other functionalized carbonized materials, compounds and compositions including the same, and methods of using the same, at least some of which are unexpected. In various embodiments, the functionalized carbon matrix has a greater concentration of functional groups than other functionalized carbonized materials. In various embodiments, Strecker degradation of the amino compound is less prevalent during the method of making the functionalized carbon matrix than predicted by others, thereby leading to a functionalized carbon matrix having a higher concentration of functional groups than expected. In various embodiments, the method can produce a functionalized carbon matrix having functionalities not possible with other methods. In various embodiments, the functionalized carbon matrix can be produced from readily available starting materials, allowing production at reduced cost compared to other methods.

In various embodiments, the functionalized carbon matrix can have a higher thermal stability than other functionalized carbonized materials. In various embodiments, the higher thermal stability can allow various functional groups, such as acidic or basic groups, to remain effective at higher temperatures and in more extreme conditions than other functionalized carbonized materials, thereby allowing more effective catalysis of a greater variety of reactions and under a greater variety of conditions than possible with other functionalized carbonized materials. In various embodiments, the functionalized carbon matrix can provide a more cost-effective method to catalyze various reactions than possible with other functionalized carbonized materials or with other catalyst materials.

In various embodiments, the Maillard reaction can be performed under basic conditions, with all or the majority of the amines deprotonated and thus acting as better nucleophiles, followed by moderate-temperature pyrolysis, thereby providing a versatile platform for one-step synthesis of functionalized highly aromatic, high-molecular weight, carbon-rich materials from inexpensive feedstocks such as glucose and taurine or other functionalized alkylamines such as ethylamines.

Various embodiments provide a facile synthesis method that can create alkane linkages between a desired functional group and a carbon matrix, which can provide more hydrothermally stable carbon catalysts. In various embodiments, a high surface area substrate such as mesoporous silica is coated with the functionalized carbon matrix to provide a more active catalyst. In various embodiments, coating the silica with a functionalized carbon layer can protect it against hydrothermal degradation. In various embodiments, a glucose-taurine (GT) colloid is developed into a more robust heterogeneous catalyst.

### BRIEF DESCRIPTION OF THE FIGURES

The drawings illustrate generally, by way of example, but not by way of limitation, various embodiments discussed in the present document.

FIG. 1 illustrates selective isotope enrichment combinations in the reactions of glucose and taurine, in accordance with various embodiments.

FIGS. 2a-c illustrate a quantitative ¹³C NMR spectra of sulfonate-functionalized carbon-rich materials made by reaction of glucose with taurine (1:1), with FIG. 2a illustrating a multiCP spectra of all carbons, FIG. 2b illustrating a direct polarization spectrum of taurine-derived carbons, and FIG. 2c illustrating a direct polarization spectrum of glucose-derived carbons, in accordance with various embodiments.

FIGS. 3a-b illustrate spectrally edited ¹³C-¹³C NMR spectra of sulfonate-functionalized carbon-rich materials made by reaction of ¹³C₆-glucose with taurine (1:1), in accordance with various embodiments.

FIG. 4 illustrates a spectrally edited ¹³C-¹³C NMR spectrum of sulfonate-functionalized carbon-rich materials made by reaction of glucose with ¹³C₂-taurine (1:1), in accordance with various embodiments.

FIG. 5 illustrates two-dimensional ¹⁵N-¹³C NMR spectra of a carbon material made from ¹³C₆-glucose and ¹⁵N-enriched taurine (1:1 molar ratio), with top portion (a) showing a spectrum of all C bonded to N, middle portion (b) showing a spectrum of nonprotonated C bonded to N, and with bottom portion (c) showing a spectrum of protonated C bonded to N, in accordance with various embodiments.

FIG. 6 illustrates a multiCP ¹⁵N NMR spectrum of a sulfonated carbon-rich material made from ¹³C glucose and ¹⁵N-enriched taurine (1:1 molar ratio), in accordance with various embodiments.

FIG. 7 illustrates a proposed structure as deduced from NMR analysis, in accordance with various embodiments.

FIG. 8 illustrates selective ¹³C NMR spectra of C bonded to N in the top two spectra and C not bonded to N in the bottom two spectra, obtained with spectral editing based on ¹³C{¹⁵N} and ¹³C{¹H} dipolar couplings, in a carbon material made from ¹³C₆-glucose and ¹⁵N-enriched taurine (1:1 molar ratio), at 7-kHz MAS, in accordance with various embodiments.

FIG. 9 illustrates the mass % of C, N, S for GTaur vs. temperature (heating for 10 hours), in accordance with various embodiments.

FIG. 10 illustrates the sulfur 2p XPS spectra of GTaur materials prepared at three temperatures, in accordance with various embodiments.

FIGS. 11a-d illustrate a quantitative multiCP ¹³C NMR spectra of functionalized carbon-rich materials made by reaction of glucose with (a) aminoethyl phosphonic acid and (b-d) aminoethylpyridine, in accordance with various embodiments.

FIG. 12 illustrates multiCP ¹³C NMR spectra of amino-piperidine 250° C. analog of melanoidin reaction synthesis, in accordance with various embodiments.

FIG. 13 illustrates the mmoles of methyl acetate formed per gram of catalyst versus time, in accordance with various embodiments.

FIG. 14 illustrates electrophoresis of GTaur on a 1% agarose gel, in accordance with various embodiments.

FIGS. 15a-b illustrate a solid-state ¹³C NMR spectrum of sulfonic-acid functionalized GT4:1 material made from glucose and taurine, with FIG. 15a showing a quantitative multiCP spectrum, and with FIG. 15b showing selective spectra of CH and CH₂ groups obtained by spectral editing, in accordance with various embodiments.

FIG. 16 illustrates elemental analyses of bulk GT catalysts, in accordance with various embodiments.

FIGS. 17a-b illustrate SEM images of GTK (17a) and GTNa (17b), in accordance with various embodiments.

FIGS. 18a-b illustrate SEM images of GT on SBA-15 (18a) and GT on Mesoporous Carbon (18b), in accordance with various embodiments.

FIG. 19 illustrates sulfur retention of the GT catalysts after repeated hydrothermal carbonization treatments at 160° C. in liquid water for 24 hours, in accordance with various embodiments.

FIG. 20 illustrates the reaction rates for various catalysts throughout the hydrothermal carbonization treatments, in accordance with various embodiments.

FIG. 21 illustrates elemental analysis data of various catalysts, in accordance with various embodiments.

FIG. 22 illustrates a SEM image of sample 1:1GT200, in accordance with various embodiments.

FIGS. 23a-g illustrate the evolution of (a) CO₂, (b) CO, (c) SO₂, (d) NH₃, (e) H₂O, (f) H₂, and (g) CH₄ during TPD analysis of aminoethanesulfonic acid containing hydrothermal carbons, in accordance with various embodiments.

FIG. 24 illustrates a proposed reaction pathway for carbon monoxide, hydrogen, and methane formation during pyrolysis of various materials, in accordance with various embodiments.

FIG. 25 illustrates an ATR-FTIR spectra of various sulfonated hydrothermal carbons, in accordance with various embodiments.

FIG. 26a-c illustrate solid state ¹³C NMRs of a 2:1GT250 sample, in accordance with various embodiments.

FIG. 27 illustrates sulfur retention data for various hydrothermal GT catalysts, in accordance with various embodiments.

FIG. 28 illustrates reaction rate date for various hydrothermal GT catalysts throughout various hydrothermal carbonization treatments, in accordance with various embodiments.

### DETAILED DESCRIPTION OF THE INVENTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods of manufacturing described herein, the steps can be carried out in any order without departing from the principles of the invention, except when a temporal or operational sequence is explicitly recited. Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

Selected substituents within the compounds described herein are present to a recursive degree. In this context, “recursive substituent” means that a substituent may recite another instance of itself or of another substituent that itself recites the first substituent. Recursive substituents are an intended aspect of the disclosed subject matter. Because of the recursive nature of such substituents, theoretically, a large number may be present in any given claim. One of ordinary skill in the art of organic chemistry understands that the total number of such substituents is reasonably limited by the desired properties of the compound intended. Such properties include, by way of example and not limitation, physical properties such as molecular weight, solubility, and practical properties such as ease of synthesis. Recursive substituents can call back on themselves any suitable number of times, such as about 1 time, about 2 times, 3, 4, 5, 6, 7, 8, 9, 10, 15, 20, 30, 50, 100, 200, 300, 400, 500, 750, 1000, 1500, 2000, 3000, 4000, 5000, 10,000, 15,000, 20,000, 30,000, 50,000, 100,000, 200,000, 500,000, 750,000, or about 1,000,000 times or more.

The term “about” as used herein can allow for a degree of variability in a value or range, for example, within 10%, within 5%, or within 1% of a stated value or of a stated limit of a range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

The term “organic group” as used herein refers to but is not limited to any carbon-containing functional group. For example, an oxygen-containing group such as an alkoxy group, aryloxy group, aralkyloxy group, oxo(carbonyl) group, a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R), SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂ N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, or C(═NOR)R wherein R can be hydrogen (in examples that include other carbon atoms) or a carbon-based moiety, and wherein the carbon-based moiety can itself be further substituted.

The term “substituted” as used herein refers to an organic group as defined herein or molecule in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxyl groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxylamines, nitriles, nitro groups, N-oxides, hydrazides, azides, and enamines; and other heteroatoms in various other groups. Non-limiting examples of substituents J that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, or C(═NOR)R wherein R can be hydrogen or a carbon-based moiety, and wherein the carbon-based moiety can itself be further substituted; for example, wherein R can be hydrogen, alkyl, acyl, cycloalkyl, aryl, aralkyl, heterocyclyl, heteroaryl, or heteroarylalkyl, wherein any alkyl, acyl, cycloalkyl, aryl, aralkyl, heterocyclyl, heteroaryl, or heteroarylalkyl or R can be independently mono- or multi-substituted with J; or wherein two R groups bonded to a nitrogen atom or to adjacent nitrogen atoms can together with the nitrogen atom or atoms form a heterocyclyl, which can be mono- or independently multi-substituted with J.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbons or, in some embodiments, from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “acyl” as used herein refers to a group containing a carbonyl moiety wherein the group is bonded via the carbonyl carbon atom. The carbonyl carbon atom is also bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. In the special case wherein the carbonyl carbon atom is bonded to a hydrogen, the group is a “formyl” group, an acyl group as the term is defined herein. An acyl group can include 0 to about 12-20 or 12-40 additional carbon atoms bonded to the carbonyl group. An acyl group can include double or triple bonds within the meaning herein. An acryloyl group is an example of an acyl group. An acyl group can also include heteroatoms within the meaning here. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “aryl” as used herein refers to cyclic aromatic hydrocarbons that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed herein.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heterocyclyl” as used herein refers to aromatic and non-aromatic ring compounds containing 3 or more ring members, of which, one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to indenyl, N-hydroxytetrazolyl, N-hydroxytriazolyl, N-hydroxyimidazolyl, indolyl, oxadiazolyl, isoxazolyl, quinazolinyl, isoindanyl, benzhydryl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include one to about 12-20 or about 12-40 carbon atoms bonded to the oxygen atom, and can further include double or triple bonds, and can also include heteroatoms. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “hydrocarbon” as used herein refers to a functional group or molecule that includes carbon and hydrogen atoms. The term can also refer to a functional group or molecule that normally includes both carbon and hydrogen atoms but wherein all the hydrogen atoms are substituted with other functional groups.

The term “number-average molecular weight” as used herein refers to the ordinary arithmetic mean of the molecular weight of individual molecules in a sample. It is defined as the total weight of all molecules in a sample divided by the total number of molecules in the sample. Experimentally, the number-average molecular weight (MO is determined by analyzing a sample divided into molecular weight fractions of species i having n, molecules of molecular weight Mᵢ through the formula Mₙ=ΣMᵢnᵢ/Σnᵢ. The number-average molecular weight can be measured by a variety of well-known methods including gel permeation chromatography, spectroscopic end group analysis, and osmometry. If unspecified, molecular weights of polymers given herein are number-average molecular weights.

The term “weight-average molecular weight” as used herein refers to M_{w}, which is equal to ΣMᵢ²nᵢ/ΣMᵢnᵢ where nᵢ is the number of molecules of molecular weight Mᵢ. In various examples, the weight-average molecular weight can be determined using light scattering, small angle neutron scattering, X-ray scattering, and sedimentation velocity.

The term “solvent” as used herein refers to a liquid that can dissolve a solid, liquid, or gas. Nonlimiting examples of solvents are silicones, organic compounds, water, alcohols, ionic liquids, and supercritical fluids.

The term “room temperature” as used herein refers to a temperature of about 15° C. to 28° C.

As used herein, the term “polymer” refers to a molecule having at least one repeating unit and can include copolymers.

### Functionalized Carbon Matrix.

In various embodiments, the present invention provides a functionalized carbon matrix having the structure:

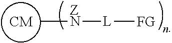

At each occurrence, Z is independently selected from H and a bond to CM. The variable CM represents the carbon matrix. The variable L represents a linker. The variable FG represents a functional group. The variable n represents the degree of substitution (e.g., the number of times CM is substituted by -L-FG).

The carbon matrix CM can be substituted or unsubstituted. The carbon matrix can be a reaction product of at least one of hydrothermal carbonization and pyrolysis of a reducing sugar and an amine having the structure H₂N-L-FG. The carbon matrix can be a reaction product of at least one of a hydrothermal carbonization and pyrolysis of a reducing sugar and the amino-group of an amine having the structure H₂N-L-FG. The carbon matrix can include the nitrogen from the amine. Thus, the functionalized carbon matrix can be represented as:

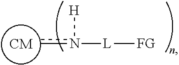

wherein — is a single or double bond to the carbon matrix, and wherein each N-L-FG unit either includes an N—H singly bonded to the CM, or an N doubly bonded to the CM and no bonded to an —H. For example, N can be single bonded to CM with —H present, or N can be doubly bonded to CM with —H absent, wherein doubly bonded can indicate a single bond to two different carbon atoms. In some embodiments, Z is a bond to the carbon matrix, and the functionalized carbon matrix can be represented as:

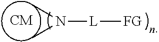

The linker L can be independently substituted or unsubstituted (C₁-C₄₀)hydrocarbyl interrupted by 0, 1, 2, or 3 atoms chosen from —S—, —O—, and —NR³—. At each occurrence L can be independently chosen from substituted or unsubstituted (C₁-C₄₀)hydrocarbyl. At each occurrence L can be independently chosen from (C₁-C₁₀)hydrocarbyl. At each occurrence L can be independently chosen from methylene, ethylene, propylene, butylene, pentylene, hexylene, heptylene, octylene, nonylene, decylene, and phenylene. At each occurrence L can be independently chosen from methylene, ethylene, butylene, and phenylene.

The functional group FG can be any suitable functional group. At each occurrence functional group FG can be independently chosen from —OR³, —OOR³, —OC(O)N(R³)₂, —CN, —CF₃, —OCF₃, —C(O), methylenedioxy, ethylenedioxy, —N(R³)₂, —SR³, —SOR³, —SO₂N(R³)₂, —C(O)R³, —C(O)C(O)R³, —C(O)CH₂C(O)R³, —C(S)R³, —C(O)OR³, —OC(O)R³, —C(O)N(R³)₂, —OC(O)N(R³)₂, —C(S)N(R³)₂, —N(R³)C(O)R³, —N(R³)N(R³)₂, —N(R³)N(R³)C(O)R³, —N(R³)N(R³)C(O)OR³, —N(R³)N(R³)CON(R³)₂, —N(R³)SO₂R³, —N(R³)SO₂N(R³)₂, —N(R³)C(O)OR³, —N(R³)C(O)R³, —N(R³)C(S)R³, —N(R³)C(O)N(R³)₂, —N(R³)C(S)N(R³)₂, —N(COR³)COR³, —N(OR³)R³, —C(═NH)N(R³)₂, —C(O)N(OR³)R³, —C(═NOR³)R³, —S(O)(OR¹)₃, —S(O)(OR¹)₂R², —S(O)(OR¹)R²₂, —S(O)R²₂, —OS(O)(OR¹)₃, —OS(O)(OR¹)₂R², —OS(O)(OR¹)R²₂, —OS(O)R²₂, —S(O)₂OR¹, —S(O)₂R², —OS(O)₂OR¹, —OS(O)₂R², —P(O)(OR¹)₂, —P(O)(OR¹)R², —P(O)R²₂, —OP(O)(OR¹)₂, —OP(O)(OR¹)R², —OP(O)R²₂, and a substituted or unsubstituted nitrogen-containing (C₁-C₂₀)heterocycle. At each occurrence R¹ can be independently chosen from —H, a counterion, and substituted or unsubstituted (C₁-C₂₀)hydrocarbyl (e.g., C₁-C₂₀ alkyl, such as C₁-C₅ alkyl). At each occurrence R² can be independently chosen from substituted or unsubstituted (C₁-C₂₀)hydrocarbyl (e.g., C₁-C₂₀ alkyl, such as C₁-C₅ alkyl). At each occurrence R³ can be independently chosen from —H and substituted or unsubstituted (C₁-C₂₀)hydrocarbyl (e.g., C₁-C₂₀ alkyl, such as C₁-C₅ alkyl).

In various embodiments, the counterion is any suitable positively charged counterion. For example, the counterion can be sodium (Na⁺), potassium (K⁺), lithium (Li⁺), hydrogen (H⁺), zinc (Zn⁺), or ammonium (NH₄⁺). In some embodiments, the counterion can have a positive charge greater than +1, which can in some embodiments be complexed with multiple ionized groups, such as Ca²⁺, Mg²⁺, Zn²⁺ or Al³⁺.

In some embodiments, FG is independently chosen from —S(O)(OR¹)₃, —S(O)(OR¹)₂R², —S(O)(OR¹)R²₂, —S(O)R²₂, —OS(O)(OR¹)₃, —OS(O)(OR¹)₂R², —OS(O)(OR¹)R²₂, —OS(O)R²₂, —S(O)₂OR¹, —S(O)₂R², —OS(O)₂OR¹, —OS(O)₂R², —P(O)(OR¹)₂, —P(O)(OR¹)R², —P(O)R²₂, —OP(O)(OR¹)₂, —OP(O)(OR¹)R², and —OP(O)R²₂. At each occurrence, FG can be independently chosen from S(O)₂OR¹ and P(O)(OR¹)₂. At each occurrence FG can be independently chosen from S(O)₂OH and P(O)(OH)₂. At each occurrence FG can be independently chosen from a substituted or unsubstituted nitrogen-containing (C₁-C₂₀)heterocycle. At each occurrence FG can be independently chosen from a substituted or unsubstituted nitrogen-containing monocyclic (C₁—O₅)heterocycle. At each occurrence FG can be independently chosen from substituted or unsubstituted pyridinyl and substituted or unsubstituted piperidinyl. At each occurrence FG can be independently chosen from pyridin-4-yl and piperidin-4-yl.

In some embodiments, FG can be independently chosen from a basic functionality, such as a nitrogen-containing heterocycle or an amine, such as pyridinyl or piperidinyl. In some embodiments, FG is independently chosen from an acidic functionality, or a salt thereof, such as S(O)₂OH and P(O)(OH)₂. In some embodiments, the functionalized carbon matrix includes at least some basic functionalities and at least some acidic functionalities.

In some embodiments, at each occurrence L is independently chosen from ethylene, butylene, methylene, and phenylene, and at each occurrence FG is independently chosen from S(O)₂OH, P(O)(OH)₂, pyridin-4-yl, and piperidin-4-yl, pyridin-2-yl, and piperidin-2-yl, pyridin-3-yl, and piperidin-3-yl.

The degree of substitution n can be about 1 to about 1,000,000, or about 1, 2, 3, 4, 5, 10, 15, 20, 25, 50, 75, 100, 125, 150, 175, 200, 225, 250, 500, 1,000, 1,250, 1,500, 2,000, 3,000, 4,000, 5,000, 7,500, 10,000, 15,000, 20,000, 25,000, 50,000, 100,000, 200,000, 500,000, or about 1,000,000 or more.

### Nanomaterials, Compositions, Composites.

In various embodiments, the present invention provides a composition or composite that includes the functionalized carbon matrix. Any suitable weight percent of the composition or composite can be the functionalized carbon matrix, such as about 0.001 wt % to about 99.999 wt %, or about 0.1 wt % to about 99.9 wt %, or about 1 wt % to about 50 wt %, or about 50 wt % to about 100 wt %, or about 0.001 wt % or less, or about 0.01 wt %, 0.1, 1, 2, 3, 4, 5, 10, 15, 20, 25, 30, 35, 40, 45, 50, 55, 60, 65, 70, 75, 80, 85, 90, 95, 96, 97, 98, 99, 99.9, 99.99, or about 99.999 wt % or more.

In various embodiments, the present invention provides a composition including the functionalized carbon matrix. In some embodiments, the composition can be a colloidal composition, such as a liquid (e.g., an aqueous liquid, an oil, an organic solvent, or a combination thereof) and the functionalized carbon material suspended in colloidal form. A colloidal solution of the functionalized carbon material can provide a template for formation of metal-coordinated materials having a core including the functionalized carbon material, such as having anionic functionalized groups thereon (e.g., acidic) and cationic metal ions coordinated thereto. A colloidal solution of the functionalized carbon material can provide a template for formation of nanomaterials. A metal-coordinated compound and a nanomaterial, having a core including the functionalized carbon matrix, are examples of compounds or composites including the functionalized carbon matrix.

In some embodiments, the present invention provides a compound including the functionalized carbon matrix and at least one metal cation coordinated thereto. The metal cation can be any suitable metal cation, such as at least one of Cu⁺, Cu²⁺, Fe²⁺, Fe³⁺, Pb²⁺, Pb⁴⁺, Sn²⁺, Sn⁴⁺, Ni²⁺, Ni³⁺, Hg₂²⁺, Hg²⁺, Cr²⁺, Cr³⁺, Mn²⁺, Mn³⁺, Co²⁺, Co³⁺, Ag⁺, Zn²⁺, Cd²⁺, Sc³⁺, Na⁺, K⁺, Mg²⁺, Pd²⁺, Pt²⁺, Li⁺, Al³⁺, Cs⁺, V²⁺, V⁴⁺, Ru³⁺, Ru⁴⁺, Rh³⁺, Ce³⁺, and Ca²⁺. In some embodiments, about 10 wt % to about 80 wt % of the compound is the one or more metal cations, or about 30 wt % to about 60 wt % of the compound, or 40 wt % or more, or about 10 wt % or less, or about 15 wt %, 20, 25, 30, 35, 40, 45, 50, 55, 60, 65, 70, 75, or about 80 wt % or more.

In various embodiments, the present invention provides a nanomaterial including the functionalized carbon matrix. The nanomaterial can be any suitable nano-sized material that includes the functionalized carbon matrix. In some examples, the nanomaterial is a nanocomposite (e.g., a composite nano-sized material including the functionalized carbon matrix). The nanomaterial can have any suitable particle size, wherein particle size is the largest dimension of the particle, such as about 0.1 nm to about 200 nm, about 5 nm to about 10 nm, or about 0.1 nm or less, or about 1 nm, 2, 3, 4, 5, 10, 15, 20, 25, 30, 35, 40, 45, 50, 60, 70, 80, 90, 100, 125, 150, 175, or about 200 nm or more.

In some examples, the nanocomposite is a ferro-magnetic material (e.g., a nanomaterial including the functionalized carbon matrix and a magnetic form of iron coordinated thereto or mixed therewith). In some embodiments, about 10 wt % to about 80 wt % of the compound is a metal, or about 30 wt % to about 60 wt % of the compound, or 40 wt % or more, or about 10 wt % or less, or about 15 wt %, 20, 25, 30, 35, 40, 45, 50, 55, 60, 65, 70, 75, or about 80 wt % or more.

In some embodiments, the present invention provides composite material including a high surface area substrate and the functionalized carbon matrix deposited thereon. The high surface area substrate can be any suitable high surface area material suitable for coating the functionalized carbon matrix thereon, such as a silica substrate, such as mesoporous silica. The high surface area substrate can have any suitable surface area, such as about 100 m²/g to about 5000 m²/g, about 300 m²/g to about 2000 m²/g, or about 100 m²/g or less, or about 200 m²/g, 300, 400, 500, 600, 700, 800, 900, 1,000, 1,250, 1,500, 1,750, or about 2,000 m²/g or more. The functionalized carbon matrix deposited on the high surface area substrate can be in any suitable form, such as nanoparticles, and such as a coating or film on the high surface area substrate. In various embodiments, the composite material can act as a catalyst (e.g., a heterogeneous catalyst). The catalyst can be resistant to hydrothermal degradation. In some embodiments, the catalyst can be a catalyst useful for catalyzing reactions of biological feedstocks. In some embodiments the composite material has applications in electronics, biomedical imaging, or high-frequency electromagnetic absorbers.

In some embodiments, a silica-material (e.g., mesoporous silica) including bonds to an -L-NH₂ group can be allowed to react with a reducing sugar in the presence of the H₂N-L-FG group to incorporate covalent bonds between the resulting functionalized carbon matrix and the high surface area substrate.

### Method of Making the Functionalized Carbon Matrix.

In various embodiments, the present invention provides a method of making the functionalized carbon matrix described herein. The method can include obtaining or providing a composition including a reducing sugar and a functionalized amine having the structure H₂N-L-FG. The method can include subjecting the composition to at least one of pyrolysis and hydrothermal carbonization. The pyrolysis or hydrothermal carbonization provides the functionalized carbon matrix. The reaction that occurs between the amine and the reducing sugar can include a Maillard reaction. In various embodiments, the present invention provides a functionalized carbon material made by an embodiment of the method.

The reducing sugar can be any suitable reducing sugar. For example, the reducing sugar can be at least one chosen from glucose, fructose, glyceraldehyde, galactose, lactose, maltose, erythrose, threose, ribose, arabinose, xylose, lyxose, allose, altrose, gulose, idose, talose, ribulose, xylulose, psicose, sorbose, tagatose, and cellobiose. The reducing sugar can be glucose. Any suitable weight percent of the composition can be the reducing sugar, such as 99.999 wt %, or about 0.1 wt % to about 99.9 wt %, or about 1 wt % to about 50 wt %, or about 50 wt % to about 100 wt %, or about 0.001 wt % or less, or about 0.01 wt %, 0.1, 1, 2, 3, 4, 5, 10, 15, 20, 25, 30, 35, 40, 45, 50, 55, 60, 65, 70, 75, 80, 85, 90, 95, 96, 97, 98, 99, 99.9, 99.99, or about 99.999 wt % or more.

The amine can be any suitable primary amine. In some embodiments, the amine is chosen from at least one aminoethyl sulfonic acid or a salt thereof, aminoethyl phosphonic acid or a salt thereof, 4-(2-aminoethyl)pyridine, and 4-aminomethylpiperidine. Any suitable weight percent of the composition can be the amine having the structure H₂N-L-FG, such as 99.999 wt %, or about 0.1 wt % to about 99.9 wt %, or about 1 wt % to about 50 wt %, or about 50 wt % to about 100 wt %, or about 0.001 wt % or less, or about 0.01 wt %, 0.1, 1, 2, 3, 4, 5, 10, 15, 20, 25, 30, 35, 40, 45, 50, 55, 60, 65, 70, 75, 80, 85, 90, 95, 96, 97, 98, 99, 99.9, 99.99, or about 99.999 wt % or more.

As used herein, “pyrolysis” refers to heating at about 100° C. to about 2000° C. in an oxygen-free, oxygen-depleted, or hydrogen environment. In some embodiments, pyrolysis can include treating with a temperature of about 200° C. to about 700° C., or about 240° C. to about 600° C., or about 200° C. or less, or about 225° C., 250, 275, 300, 325, 350, 375, 400, 425, 450, 475, 500, 525, 550, 575, 600, 625, 650, 675, or about 700° C. or more. The pyrolysis can be performed for any suitable period of time, such as about 1 minute to about 7 days, or about 1 hour to about 2 days, or about 1 minute or less, or about 2 minutes, 3, 4, 5, 10, 15, 20, 30, 40, 50 minutes, 1 h, 2, 3, 4, 5, 6, 7, 8, 9, 10, 11, 12, 13, 14, 15, 16, 17, 18, 19, 20, 21, 22, 23, 24 h, 1.5 d, 2, 3, 4, 5, 6, or about 7 d or more. The environment used for the pyrolysis can have any suitable vol % of oxygen, such as about 19 vol %, 18, 17, 16, 15, 14, 13, 12, 11, 10, 9, 8, 7, 6, 5, 4, 3, 2, 1, 0.1, 0.01, 0.001, or about 0.000, 1 vol % or less. The pressure used for the pyrolysis can be any suitable pressure, such as about 0.01 bar to about 1000 bar, about 0.01 bar or less, or about 0.1, 1, 2, 3, 4, 5, 6, 8, 10, 12, 14, 16, 18, 20, 22, 24, 25, 30, 35, 40, 45, 50, 55, 60, 65, 70, 75, 80, 85, 90, 95, 100, 125, 150, 175, 200, 250, 500, 750, or about 1000 bar or more.

As used herein, “hydrothermal treatment” or “hydrothermal carbonization” refers to heating at about 110° C. to about 700° C. in water under a pressure of about 1.2 bar to about 1000 bar. In some embodiments, the hydrothermal carbonization can include treating with a temperature of about 80° C. to about 350° C., or about 90° C. to about 150° C., or about 160° C. to about 220° C., or about 80° C. or less, or about 90° C., 100, 110, 120, 130, 140, 150, 160, 170, 180, 190, 200, 210, 220, 230, 240, 250, 260, 270, 280, 290, 300, 310, 320, 330, 340, or about 350° C. or more. In some embodiments, the hydrothermal carbonization can include treating with a pressure of about 1.2 bar to about 1000 bar, about 1.2 bar or less, or about 1.4, 1.6, 1.8, 2, 2.5, 3, 3.5, 4, 4.5, 5, 6, 8, 10, 12, 14, 16, 18, 20, 22, 24, 25, 30, 35, 40, 45, 50, 55, 60, 65, 70, 75, 80, 85, 90, 95, 100, 125, 150, 175, 200, 250, 500, 750, or about 1000 bar or more. The hydrothermal carbonization can be performed for any suitable period of time, such as about 1 minute to about 7 days, or about 1 hour to about 2 days, or about 1 minute or less, or about 2 minutes, 3, 4, 5, 10, 15, 20, 30, 40, 50 minutes, 1 h, 2, 3, 4, 5, 6, 7, 8, 9, 10, 11, 12, 13, 14, 15, 16, 17, 18, 19, 20, 21, 22, 23, 24 h, 1.5 d, 2, 3, 4, 5, 6, or about 7 d or more.

In some embodiments, the method further includes treating with a temperature of about 30° C. to about 120° C. (e.g., 30° C. or less, or about 40° C., 50, 60, 70, 80, 90, 100, 110, or about 120° C. or more) for about 30 minutes to about 14 days (e.g., 30 minutes or less, or about 1 h, 2, 3, 4, 5, 6, 7, 8, 9, 10, 11, 12, 13, 14, 15, 16, 17, 18, 19, 20, 21, 22, 23, 24 h, 1.5 d, 2, 3, 4, 5, 6, 7, 8, 9, 10, 11, 12, 13, or about 14 d or more) prior to the hydrothermal treatment or pyrolysis. For example, when performing the method using volatile starting materials (e.g., various amines), it can be useful to initiate reaction between the sugar and the amine for a period of time before increasing the temperature, otherwise the volatile starting material may evaporate without reacting.

In some embodiments, the composition further includes a base. The base can be any suitable base, such as an alkali based or an organic base, such as at least one of NaOH, KOH, NaHCO₃, Na₂CO₃, Ca(OH)₂, and LiOH.

The method can further include making a colloidal mixture including the functionalized carbon matrix and a fluid. The method can include coordinating at least one metal cation to the functionalized carbon matrix. The method can include templating a nanocomposite with the functionalized carbon matrix.

### Method of Making a Nitrogen-Rich Carbon Material.

In various embodiments, the present invention provides a method of making a nitrogen-rich carbon material. The method can include obtaining or providing a composition including a reducing sugar and a functionalized amine having the structure H₂N-L-FG. The method can include subjecting the composition to pyrolysis including a temperature of about 500° C. to about 2000° C., to provide a nitrogen-rich carbon material. In various embodiments, the present invention can provide a nitrogen-rich carbon material made by an embodiment of the method. In various embodiments, the nitrogen-rich carbon materials can have applications such as catalysts, contrast agents for biomedical imaging, high-frequency electromagnetic absorbers, and magnetic-fluid hyperthermia cancer therapy.

The nitrogen-rich carbon material can have any suitable weight percent nitrogen, such as about 10 wt % to about 40 wt %, or about 10 wt % or less, or about 11 wt %, 12, 13, 14, 15, 16, 17, 18, 19, 20, 22, 24, 26, 28, 30, 32, 34, 36, 38, or about 40 wt % or more.

### Examples

Various embodiments of the present invention can be better understood by reference to the following Examples which are offered by way of illustration. The present invention is not limited to the Examples given herein.

Part I. A Simple One-Step Synthesis of Carbon-Rich Materials with High Concentrations of Stable Catalytic Sites, Validated by NMR.

Summary. A facile one-step synthesis of carbon-rich highly aromatic materials containing alkyl-linked Brønsted acidic or basic groups is described. It is based on the Maillard reaction and low-temperature (250° C.) pyrolysis of glucose with primary amines that contain acidic sulfonic or phosphonic functionalites, or basic piperidine or pyridine groups. The resulting black materials were characterized by one- and two-dimensional solid-state ¹³C and ¹⁵N NMR, supplemented by elemental analysis. Synthesis with ¹³C-enriched glucose enabled a selective and comprehensive NMR characterization of the aromatic scaffold, which was composed mostly of C_{α}-linked pyrrole, indole, and some pyridine aromatic rings with ketone linkers and CH₃ pendent groups. The matrix aromaticity is 60% for the amino ethyl sulfonate material. Distinct CH₂ groups bonded to heteroatoms confirmed that the R-functional groups mostly remain intact at 250° C. The linkages resulting from reaction of the amine and glucose were explored by ¹⁵N-¹³C, ¹³C{¹⁵N}, and multiCP ¹⁵N of a material made from ¹⁵N-taurine with ¹³C-enriched glucose. ¹⁵N NMR shows that no unreacted alkyl amine groups remain in the material. The N-ethyl-R groups are incorporated mostly through pyrrole rings, half of which are part of indoles; some secondary and tertiary amines and amides, as well as pyridines, are also observed. The materials were catalytically active for esterification reactions.

Elemental and XPS Analysis.

Elemental analyses of materials in terms of mass % of carbon, hydrogen, nitrogen, and sulfur (CHNS) were performed using a PE 2100 Series II combustion analyzer (Perkin Elmer Inc., Waltham, Mass.). The chemical state of sulfur was analyzed using a Physical Electronics 550 Multitechnique XPS system employing a standard Al electron source. The samples were run at 10⁻⁹ Torr and mounted on double-sided tape. Charging was corrected for by adjusting the carbon peak to 284 eV.

Quantitative ¹³C NMR.

Quantitative ¹³C spectra were obtained on labeled materials using direct polarization (DP) with 45 s recycle delays, and multiCP on unlabeled materials. Given the low signal-to-noise ratio, and longer relaxation times, DP experiments on the unlabeled materials are very time consuming. The recently introduced multiCP sequence solved this problem, showing excellent agreement with DP spectra for a wide variety of materials, including low to moderate temperature carbon materials. The experiments used five 1.1-ms CP periods and repolarization delays of 0.9 s under 14 kHz magic-angle spinning (MAS). DP experiments used a 4.3 μs ¹³C 90° pulse length, with 65 kHz ¹H decoupling during acquisition following a Hahn-echo to refocus ¹³C magnetization. Corresponding spectra of nonprotonated C and mobile segments (including CH₃) were obtained after 68 μs of recoupled ¹H-¹³C dipolar dephasing before detection [Mao & Schmidt-Rohr].

2D ¹⁵N-¹³C NMR.

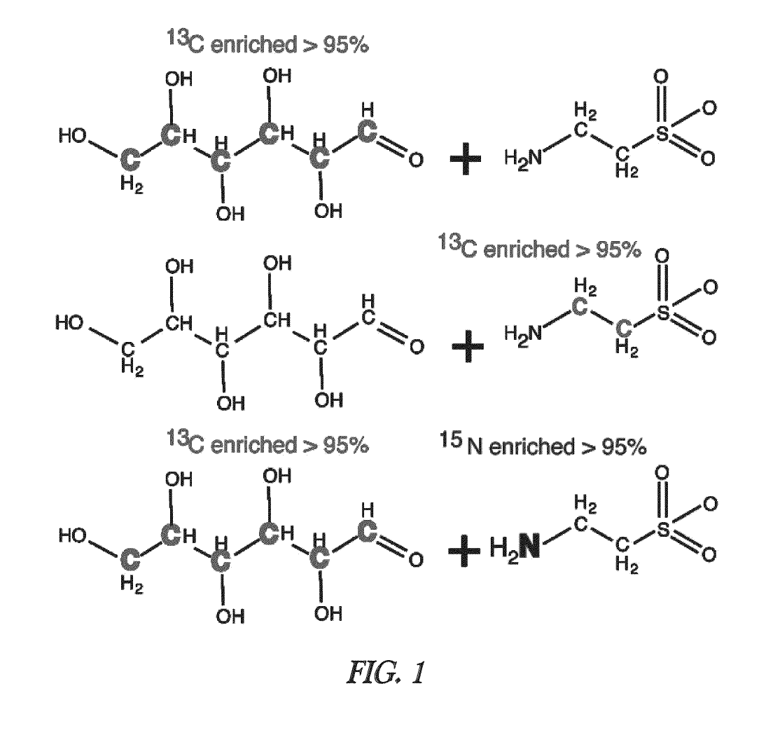

In order to detect the connectivities between glucose-¹³C and taurine-¹⁵N on GTaur materials produced using the isotopic labeling in FIG. 1, the 2D ¹⁵N-¹³C heteronuclear single quantum (HSQC) pulse sequence with REDOR recoupling was applied, at a MAS frequency of 7 kHz with 4-pulse total suppression of spinning sidebands (TOSS) applied before detection. The total experiment time was 18 h per 2D spectrum.

MultiCP ¹⁵N NMR.

Nearly quantitative ¹⁵N NMR spectra were obtained using multiCP with five CP periods of 5.5-ms total duration, at 7 kHz MAS, with a Hahn spin echo before detection. The ¹⁵N 90° pulse length was 10 μs. A selective multiCP spectrum of nonprotonated N was obtained by ¹H-¹⁵N dipolar dephasing of 2 tᵣ duration before detection, with recoupling of the heteronuclear dipolar interactions by two ¹H 180° pulses. Partial dephasing of the nonprotonated nitrogen was corrected for by multiplication with 1.3, the correction factor determined in a ¹⁵N-acetyl proline model compound. In addition, the intensity of the nonprotonated N relative to that of NH was scaled up by 10%, to compensate for incomplete cross polarization of nonprotonated N.

Combined ¹³C{¹⁵N} and ¹³C{¹H} Spectral Editing.

Signals of C bonded to N, and their complement, peaks of C not bonded to N, were selected by ¹³C{¹⁵N} REDOR of Ntᵣ= 1/7 k ms duration. Spectra recorded with (S) and without (S₀) 180° pulses on the ¹⁵N channel contain signals mostly of C not bonded to N and the full reference signal S₀, respectively. The difference ΔS=S₀−S derives selectively from C bonded to N, with <10% contributions from C two bonds from N. A spectrum purely of C not bonded to N, without residual C—N signals, was obtained as S′=S−0.15 ΔS. The experiments were performed at 7 kHz MAS with TOSS before detection.

Selection of CHₙ signals was achieved by short (80 μs) CP combined with 40-μs dipolar-dephasing difference, which in combination with ¹³C{¹⁵N} REDOR yielded selective spectra of NCHₙ (ΔS) and of CHₙ not bonded to N (S−0.15 ΔS). Conversely, signals from nonprotonated carbons and mobile segments were selected by 40 μs of dipolar dephasing, and combined with ¹³C{¹⁵N} REDOR to give selective spectra of nonprotonated C bonded to N and of nonprotonated or mobile C not bonded to N.

Spectrally edited 2D ¹³C-¹³C NMR.

Two-dimensional (2D) NMR spectra to identify nonprotonated C near CH were measured by 2D exchange NMR after short (0.07 ms) cross-polarization and with dipolar dephasing before detection (exchange with protonated and nonprotonated spectral editing, EXPANSE NMR), at a MAS frequency of υᵣ=14 kHz. Dipolar assisted rotary recoupling (DARR) was applied during the mixing time of 10 ms. Structural models were generated for each of the sulfonated carbon materials based on the quantitative 1D and spectrally edited 2D ¹³C NMR spectra as well as elemental analysis (C, H, N, S, and then O by difference), which provide relative amounts of key structural fragments and atomic compositions and thus greatly constrain the possible structures. The spectrally edited two-dimensional NMR experiments were used to identify the predominant linkage patterns of the aromatic subunits and the ketones.

Catalytic Activity.

Catalyst performance was tested by ester formation of acetic acid and methanol, which it is sensitive to the number of acidic sites and their pKₐ value. Reactions were run at 40° C. in a 10-mL Alltech reactor that was vial loaded with 6 M methanol and 3 M acetic acid in dioxane (total volume 7 mL) and 10 mg of the sulfonated material. By running the reactions at low conversions (<5%), initial rates could be compared. Tests with a range of stir rates showed that no mass transfer effects occurred for stir rates >200 rpm. The products were analyzed using an Agilent 7890A gas chromatograph with a flame-ionization detector. The activity per mass of catalyst was compared, with units of mmol acetate formed min⁻¹ (g catalyst)⁻¹.

### Example 1

### Preparation of Samples

Glucose and each of the selected amines (aminoethyl sulfonic acid, aminoethyl phosphonic acid, 4-(2-aminoethyl)pyridine and 4-aminomethylpiperidine) were co-precipitated from aqueous solution, freeze-dried, and pyrolyzed in a MTI 1200X tube furnace with a 1° C./minute heating ramp in flowing ultra-pure Argon gas passed through a gettering furnace and reacted for 10 h at the final temperature (250° C., 350° C., or 550° C.). Sulfonic-acid and phosphonic acid groups were introduced by reacting glucose with taurine (‘GTaur’) or aminoethyl phosphonic acid (‘GPhos’) and KOH in a 1:1:1 ratio. The reaction of glucose with aminoethylpyridine (‘GPyri’) did not require base since the primary amine was in the deprotonated form as received. Given the low boiling point of aminoethylpyridine, this reaction was carried out in two steps. In the first step, the sample was heated to 80° C. and held there for 4 h, before heating to the final temperature. After samples had cooled to room temperature, they were removed from the reactor, ground with mortar and pestle, washed with e-pure water, and dried in a vacuum oven at 110° C. Materials produced with sulfonic functional groups dispersed into a colloidal phase with exposure to water, and were washed in 0.5 M HCl in ethanol instead. To probe the fate of the different reactants, three different selective ¹³C and ¹⁵N-labeling combinations for the GT materials were used, as shown in FIG. 1, with ¹³C and ¹⁵N are indicated by bold letters. By using ¹⁵N-labeled taurine and uniformly ¹³C-labeled glucose, it is straightforward to determine the structures of the N—C linkages with 2D ¹³C-¹⁵N NMR experiments.

### Example 2

### ¹³C NMR of Glucose-Taurine Materials

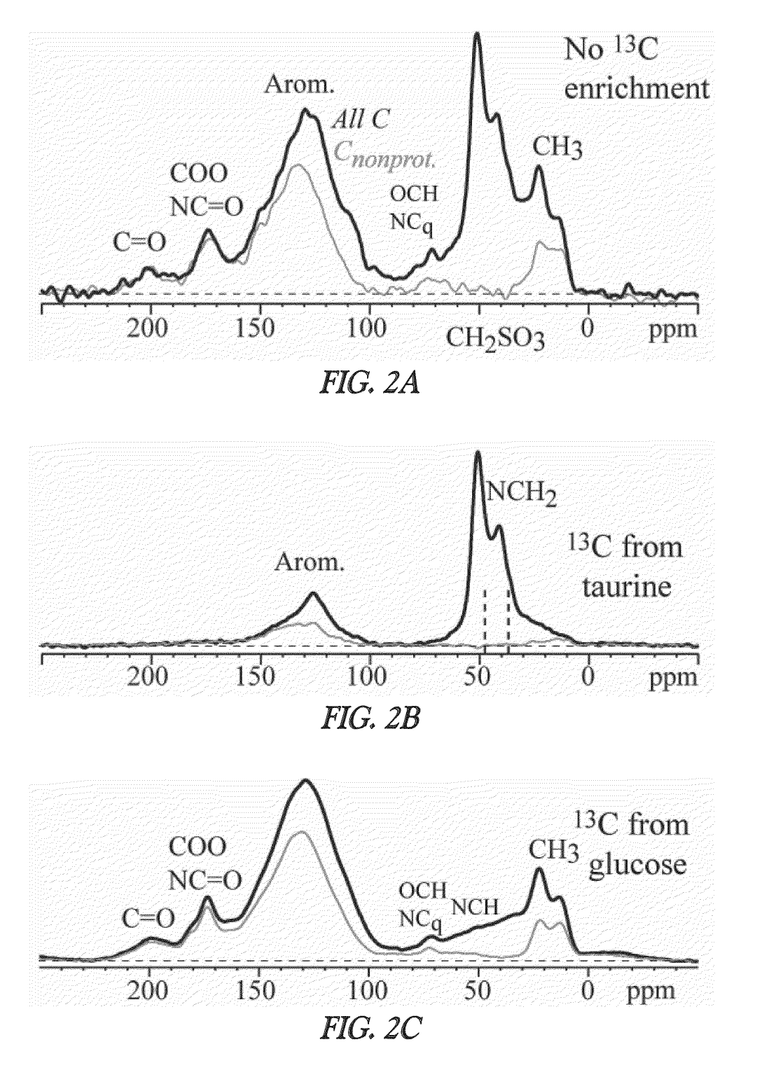

Shown in FIGS. 2a-c are quantitative ¹³C NMR spectra for GT materials produced at 250° C. without enrichment, with ¹³C₂ taurine, and with ¹³C₆ glucose, respectively. FIG. 2 illustrates a quantitative ¹³C NMR spectra of sulfonate-functionalized carbon-rich materials made by reaction of glucose with taurine (1:1). The corresponding spectra of nonprotonated C and mobile segments are shown as grey lower lines. FIG. 2a shows a multiCP spectra of all carbons (¹³C in natural abundance). FIG. 2b shows a direct polarization spectrum of taurine-derived carbons (¹³C₂ taurine); the peak positions of crystalline taurine are indicated by dashed lines. FIG. 2c shows a direct polarization spectrum of glucose-derived carbons (¹³C₆ glucose). The spectrum with ¹³C in natural abundance shows the signals of carbons originating from both glucose and taurine, while the 90-fold isotopic enrichment of one component provides selective spectra of the carbons originating from that component. As predicted, the spectrum of all carbons in (a) can be matched by a weighted superposition of the selective spectra of taurine- and glucose-derived carbons in (b) and (c), respectively.

The selective spectrum of C originating from taurine is shown in FIG. 2b. It shows two prominent peaks, at 53 and 42 ppm, which account for 60% of the total intensity and match the resonance positions of the CH₂ groups bonded to S and N, respectively, in taurine. This proves that a large fraction of taurine is incorporated with the N—CH₂—CH₂—SO₃ side group intact. The shoulder at <35 ppm is from CH₂ and CH₃ groups not bonded to heteroatoms, which must be regarded as degradation products. The only other significant signal observed is from aromatic carbons (27% of the spectral area after correction for natural-abundance background). Some of these aromatic carbons are bonded to —CH₂—SO₃ functional side groups.

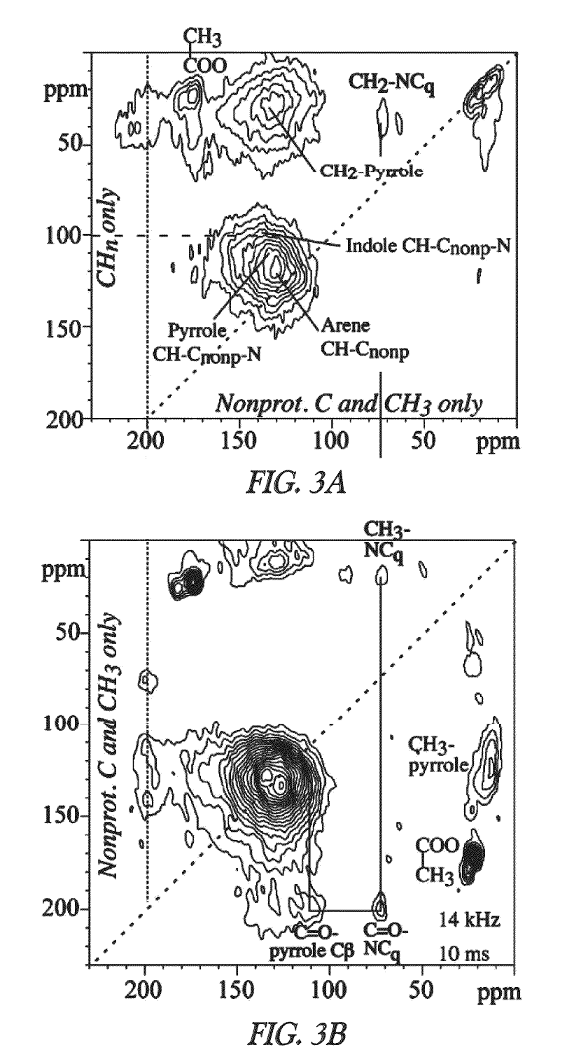

The most prominent signal of the carbons derived from glucose, see FIG. 2c, is from aromatic rings, between 150 and 100 ppm. Unlike Maillard-reactions products produced at lower temperatures, the present samples contain hardly any residual OCH or OCH₂ groups. CH₃ groups bonded to C resonating at <28 ppm show two fairly strong peaks, at 23 and 12 ppm, which can be assigned to acetyl or N-acetyl and to pyrrole-bonded CH₃, respectively. These assignments have been confirmed by corresponding cross peaks in ¹³C-¹³C NMR, see FIG. 3a-d. FIGS. 3a and b illustrate spectrally edited ¹³C-¹³C NMR spectra of sulfonate-functionalized carbon-rich materials made by reaction of ¹³C₆-glucose with taurine (1:1).

These data show that over 60% of the CH₂ groups originating from taurine are retained, resonating at chemical shifts characteristic of the sulfonic acid fragment, and more than 72% of the glucose has been converted to sp²-hybridized carbons. Results from XPS and CHNS elemental analysis corroborate the NMR findings, and verify that the sulfur is retained and remains predominantly in the desired C—SO₃H form. Integration of the one-dimensional ¹³C NMR spectra directly provided quantitative amounts of major functional groups present, including ketones, carboxylic acids, protonated and nonprotonated aromatic C, O-alkyl and nonpolar alkyl C, see Table 1.

TABLE 1
Peak areas (in %) from GTaur ¹³C spectra.

| | Integration limits (ppm) | | | | | |
|---|---|---|---|---|---|---|
| | 220-185 | 185-162 | 162-96 | 96-65 | 65-29 | 29-0 |
| No ¹³C enrichment All C | 3 | 6 | 44 | 5 | 31 | 12 |
| Cₙₒₙₚᵣₒₜ | 3 | 6 | 27 | 1 | 1 | 5 |
| >95% ¹³C taurine All C | 1 | 2 | 28 | 2 | 58 | 8 |
| Cₙₒₙₚᵣₒₜ | 0 | 1 | 15 | 0 | 2 | 2 |
| >95% ¹³C glucose All C | 4 | 8 | 61 | 4 | 10 | 13 |
| Cₙₒₙₚᵣₒₜ | 3 | 7 | 40 | 2 | 1 | 6 |

### Example 3

### Aromatic Bond Formation from Taurine

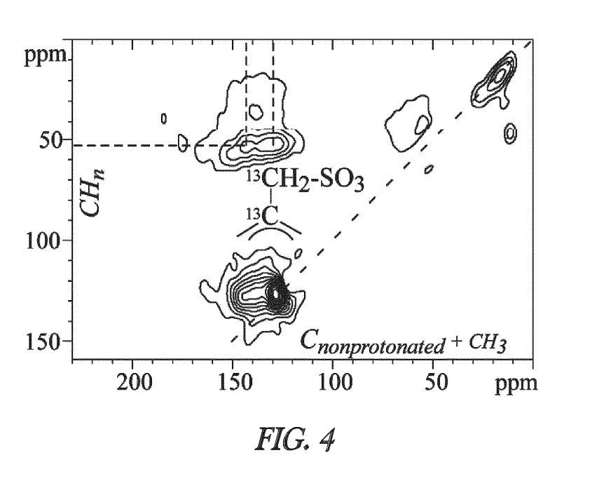

Considering that nearly 27% of the CH₂ groups on taurine are converted to aromatic carbons, experiments were conducted to probe the structures formed. The cross peaks of the aromatic carbons in the 2D ¹³C-¹³C NMR spectrum of FIG. 4 show that many taurine molecules are incorporated into aromatic rings without breaking the ¹³C-¹³C bond. Most interestingly, the pronounced aromatic-alkyl cross peak in the upper middle of the spectrum must be assigned to Cₐᵣₒₘ—CH₂—SO₃ moieties based on the CH₂ chemical shift near 53 ppm, which requires bonding of CH₂ to a heteroatom. The assignment to CH₂—SO₃ is confirmed by the absence of CH₂—NH₂ or CH₂—NH₃⁺ signals from the ¹⁵N spectrum shown below, and by the chemical shift of CH₂—O carbons being significantly different, >60 ppm. The CH₂—SO₃ group of the Cₐᵣₒₘ—CH₂—SO₃ moiety contributes to the larger intensity of the 53-ppm CH₂ peak in FIGS. 2a and b, which is mostly from CH₂—SO₃, while there is no contribution to the smaller N—CH₂ signal at 41 ppm, as a result of the incorporation of the taurine N—CH₂ carbon into the aromatic ring.

The aromatic chemical shifts between 130 and 150 ppm are consistent with bonding of the aromatic carbon to N in pyrrole or pyridine rings, which would leave the S—C—C—N backbone bonds of taurine intact. The Cₐᵣₒₘ—CH₂—SO₃ structures can be expected to be particularly hydrothermally stable. The interaromatic cross peak at 128 ppm CH and 135-145 ppm Cₙₒₙₚ resonance positions can be assigned to CH═Cₙₒₙₚ—N— in pyridine rings, where loss of the SO₃ group seems likely, or to arene CH═Cₙₒₙₚ—SO₃.

### Example 4

### ¹⁵N-Based NMR of Glucose-taurine Materials

To get detailed information about the incorporation of the amine group of the bifunctional molecules into the glucose-derived matrix, the chemical environment of the nitrogen atoms after reaction can be determined by ¹⁵N-¹³C, ¹³C{¹⁵N}, and ¹⁵N NMR of a material made by reacting ¹⁵N-taurine and ¹³C₆-glucose at 250° C., as described in the following. Initially 2D ¹⁵N-¹³C NMR was used, which can best identify the heterocycles and functional groups into which N is incorporated, and then estimate their amounts from one-dimensional ¹⁵N and ¹³C{¹⁵N} spectra.

### Example 4a

### Identification of Nitrogen Attachments

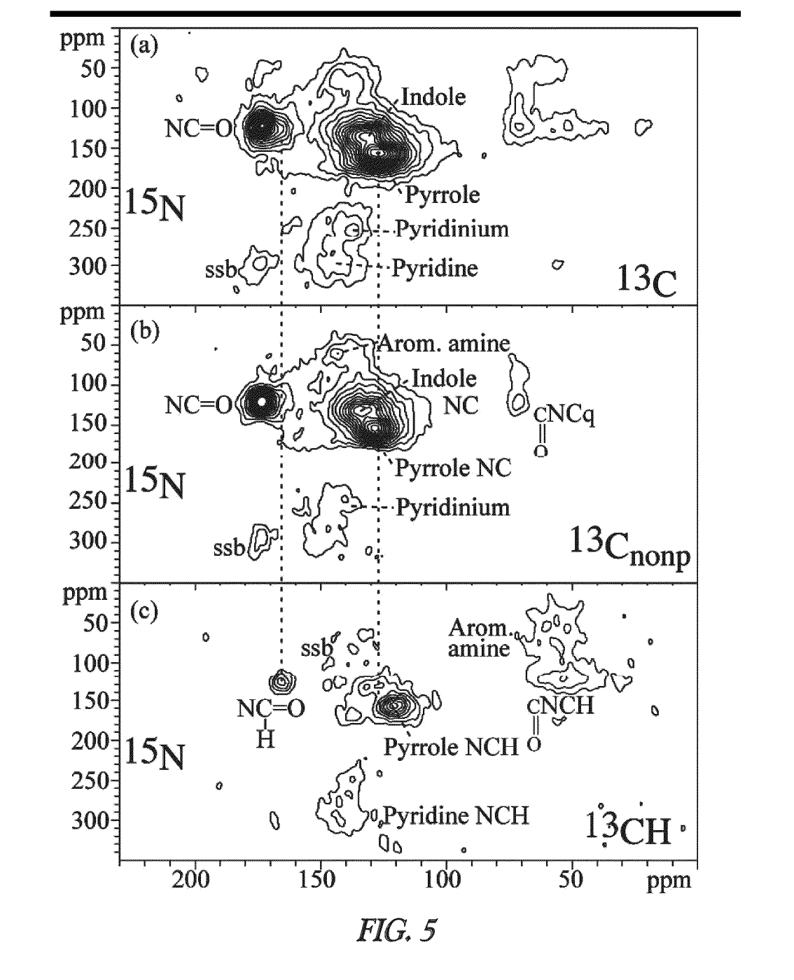

The incorporation of taurine N into the glucose-derived aromatic-rich matrix can be explored by 2D ¹³C-¹⁵N correlation NMR. FIG. 5 illustrates 2D ¹⁵N-¹³C NMR spectra of a carbon material made from ¹³C₆-glucose and ¹⁵N-enriched taurine (1:1 molar ratio), at 7-kHz MAS, with top portion (a) showing a spectrum of all C bonded to N, middle portion (b) showing a spectrum of nonprotonated C bonded to N, selected by gated decoupling before detection, and with bottom portion (c) showing a spectrum of protonated C bonded to N, selected by short CP and dipolar-dephasing difference. Among the three largest peaks in the ¹⁵N-¹³C 2D spectrum, see FIG. 5, top portion (a), two are from nitrogen bonded to aromatic carbons, according to the ¹³C chemical shifts of 125 and 135 ppm. Based on the ¹⁵N chemical shifts of 155 and 135 ppm, the peaks can be assigned to pyrrole and indole (benzopyrrole), respectively. Spectral editing of the ¹⁵N-¹³C spectrum based on ¹H-¹³C dipolar couplings, see FIG. 5 middle and bottom portions (b) and (c), shows that the C—N carbons assigned to the indole ring are less protonated than those of pyrrole. This is expected since in indole, one C—N carbon is in the bridgehead to the aromatic ring and cannot be protonated. Signal of amide, NC═O, is clearly observed near 120 ppm in the ¹⁵N and 175 ppm in the ¹³C dimension. Cross peaks between 70 and 30 ppm show that some amide N is bonded to alkyl C, both protonated and nonprotonated. A fairly small but distinctive signal of N(C—H)═O is seen near 165 in the ¹³C dimension and can be assigned to a formamide moiety.

Smaller peaks in the ¹⁵N-¹³C spectrum include signal at 250 ppm ¹⁵N and 142 ppm ¹³C. The chemical shift would match that of the second N (not bonded to H or C outside the ring) in an imidazole ring, or that of a pyridinium nitrogen bonded to three carbons. The latter assignment is much more probable given the >135 ppm ¹³C chemical shifts of these CH groups and the <130 ppm resonance position of CH in imidazole [histidine]. A broad secondary maximum seen near 300 ppm ¹⁵N and 145 ppm ¹³C can be assigned to pyridine-like N bonded to pyridinic CH and nonprotonated C.

In addition, signals from amines are observed between 100 and 50 ppm in the ¹⁵N dimension. The cross peaks show that these nitrogen atoms are bonded to aromatic C, which is also consistent with the ¹⁵N chemical shift, since alkyl-bonded amine N resonates at <50 ppm. Herein, evidence is given that shows that these amines are mostly tertiary (bonded to three C atoms), so the alkyl carbon resonances also observed in this ¹⁵N chemical shift range are from alkyl bonding partners. In other words, many of these amines link an aromatic ring to two alkyl carbons, one originating from glucose and the other from taurine.

### Example 4b

### Quantification of N Attachments, and Tertiary Vs. Secondary N

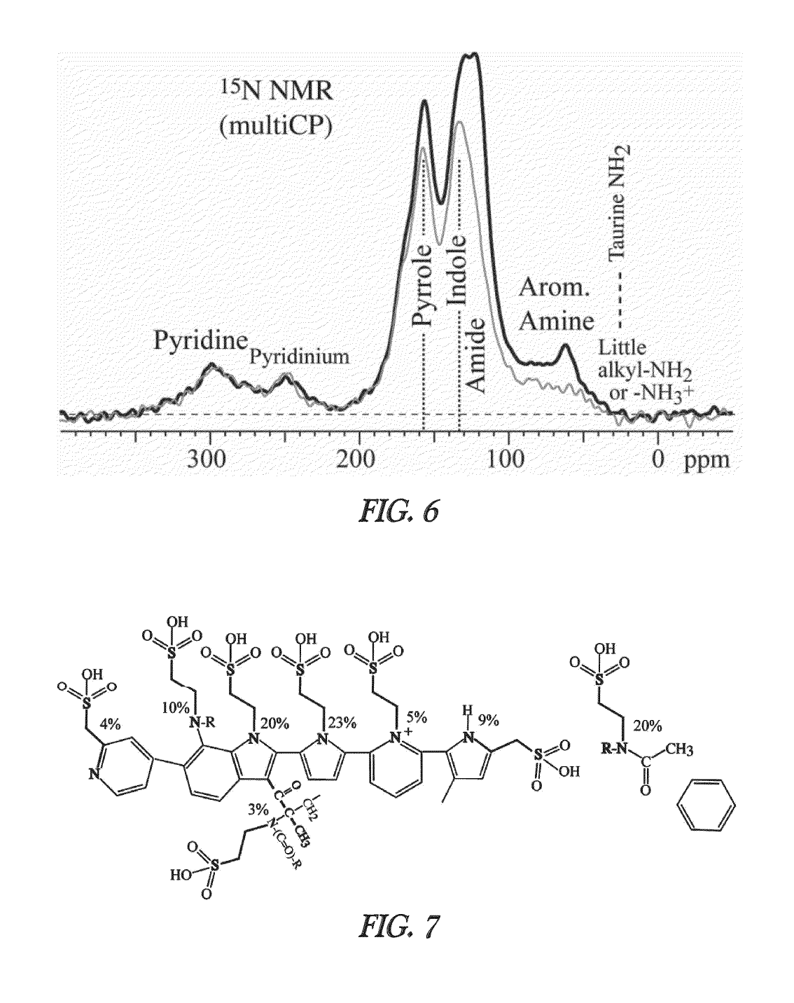

The fractions of the different types of nitrogen can be determined by analyzing peaks in nearly quantitative multiCP ¹⁵N NMR spectra, see FIG. 6. FIG. 6 illustrates a multiCP ¹⁵N NMR spectra of a sulfonated carbon-rich material made from ¹³C glucose and ¹⁵N-enriched taurine (1:1 molar ratio), at 7-kHz MAS. Black lines: Spectrum of all nitrogen. Grey lines: corresponding spectra of nonprotonated N, obtained after 280 μs of recoupled dipolar dephasing, scaled up by a dephasing factor of 1.3 determined in a model compound, ¹⁵N-tBOC proline. The frequency position of taurine is indicated. Corresponding spectra of nonprotonated nitrogen (grey line in FIG. 6), selected by 280 μs of recoupled dipolar dephasing, allow determination of the number of carbons to which they are bonded. Table 2 shows the integral values for ¹⁵N multiCP spectra of GTaur 250 material which indicate: 8% pyridine; 5% pyridinium; 20% indole; 23% pyrrole N; 12% pyrrole NH; 22% amide; and 10% amine.

TABLE 2
Integral values for ¹⁵N multiCP spectra of GTaur 250 material.

| Integral (ppm) | 350-267 | 267-212 | 212-141 | 141-91 | 91-0 |
|---|---|---|---|---|---|
| All ¹⁵N | 8 | 5 | 35 | 42 | 10 |
| ¹⁵Nₙₒₙₚᵣₒₜ | 7 | 4 | 23 | 24 | 4 |

The ¹⁵N spectrum exhibits no NH₂ signal near 40 ppm from unreacted taurine, or from taurine incorporated only via reaction of its sulfonate group. Based on the ¹⁵N-¹³C spectra, the most intense peaks in the 250° C. spectrum, at 158 and 133 ppm, can be assigned to pyrrole and indole nitrogen. The limited dipolar dephasing shows that most of these nitrogen atoms are not bonded to hydrogen, which means that they must be bonded to three carbon atoms; one of these bonds is usually the carbon already bonded to N in the precursor, in this case CH₂ of taurine. Other smaller peaks in the ¹⁵N spectrum include signal from amines between 50 and 90 ppm, most of which must be tertiary since they are not protonated. Their chemical shift indicates bonding to aromatic C, as confirmed in the ¹⁵N-¹³C spectrum. Note that no alkyl-NH₂ or alkyl-NH₃⁺ groups, which would resonate at <50 ppm, are observed.

Integration of the peaks of the ¹⁵N multiCP spectra shows that tertiary N, e.g., with the linkage to taurine C, intact accounts for about half of all N, with 23% in pyrrole, ca. 20% in indole, and 8% in tertiary amides and amines. In addition, 20% of the N was shown to be in pyrrole with NH and in pyridine rings with —CH₂—SO₃ side groups, so nearly 60% of all taurine molecules produce CH₂—SO₃ groups linked to aromatic rings. An additional 24% of the N is incorporated into secondary amides and amines, where the linkage to taurine may also be intact. FIG. 7 displays the main linkages deduced here.

### Example 4c

### Identification of Glucose C Bonded to N, and of Arene Signals

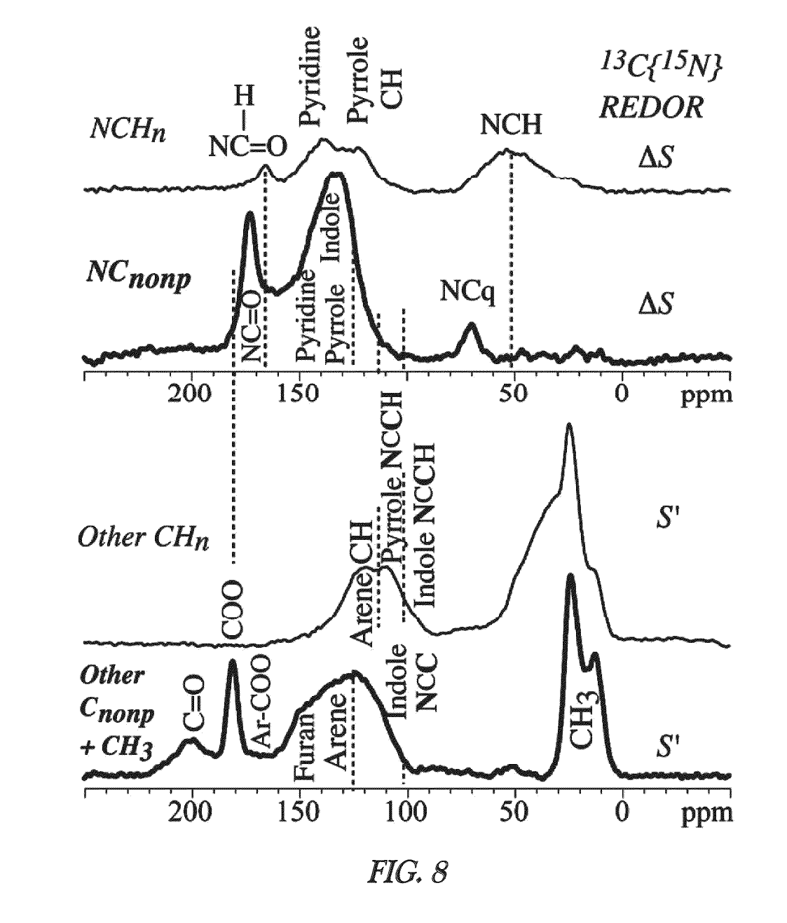

In order to identify and quantify glucose-derived C bonded to N and its complement, glucose-C not bonded to N, their signals were recorded selectively using ¹³C{¹⁵N} REDOR NMR applied to a material made from ¹³C-enriched glucose and ¹⁵N-enriched taurine. For additional deconvolution and structural information, this ¹³C{¹⁵N} dephasing was combined with ¹³C{¹H} dipolar dephasing, and with short CP plus dipolar dephasing difference. This yields selective spectra of NCHₙ, of nonprotonated C bonded to N, of CH not bonded to N, and of nonprotonated C not bonded to N, see FIG. 8, with selective ¹³C NMR spectra of C bonded to N in the top two spectra and C not bonded to N in the bottom two spectra, obtained with spectral editing based on ¹³C{¹⁵N} and ¹³C{¹H} dipolar couplings, in a carbon material made from ¹³C₆-glucose and ¹⁵N-enriched taurine (1:1 molar ratio), at 7-kHz MAS. FIG. 8, top two spectra, shows signals of C bonded to N. Top trace: NCHₙ; bottom trace: Nonprotonated C bonded to N. FIG. 8, bottom two spectra, shows signals of C not bonded to N. Top trace: CHₙ not bonded to N. Bottom trace: Nonprotonated C not bonded to N. Assignments based on the spectral editing, ¹⁵N-¹³C spectra of FIG. 5, and general ¹³C chemical shift trends are indicated. As in the ¹⁵N and the unselective ¹³C spectra, signals from aromatic rings are the most pronounced in the C—N spectra, see FIG. 5 top portion (a), indicating incorporation of the amine compound into the glucose-derived aromatic matrix.

The four spectra in FIG. 8 are quite distinctive and resolve the broad aromatic band of the unselective ¹³C spectrum in FIG. 2c into at least 4 peaks. In particular, this enables us to separate the strongly overlapping signals of NC in pyrroles in the top two specta and from those of six-membered arene rings in the bottom two spectra. In indoles, carbon 3, which is two bonds from N, has a characteristic downfield chemical shift near 100 ppm (protonated C) or near 110 ppm (nonprotonated C), according to chemical-shift databases. Not being bonded to N, this carbon should give signal in FIG. 8, bottom two spectra. Indeed, aromatic CH signal is clearly observed around 100 ppm, and Cₙₒₙₚ signal near 110 ppm.

The sharp peak at 173 ppm is from amide C═O bonded to alkyl or arene carbons, as confirmed by cross peaks in ¹³C-¹³C correlation spectra. Amide C═O bonded to pyrrole rings resonates between 160 and 170 ppm, and can explain the relatively high intensity in that range in FIG. 8, at the trace second from the top.

### Example 5

### NMR, Elemental and XPS Analysis of Pyrolysis Effects

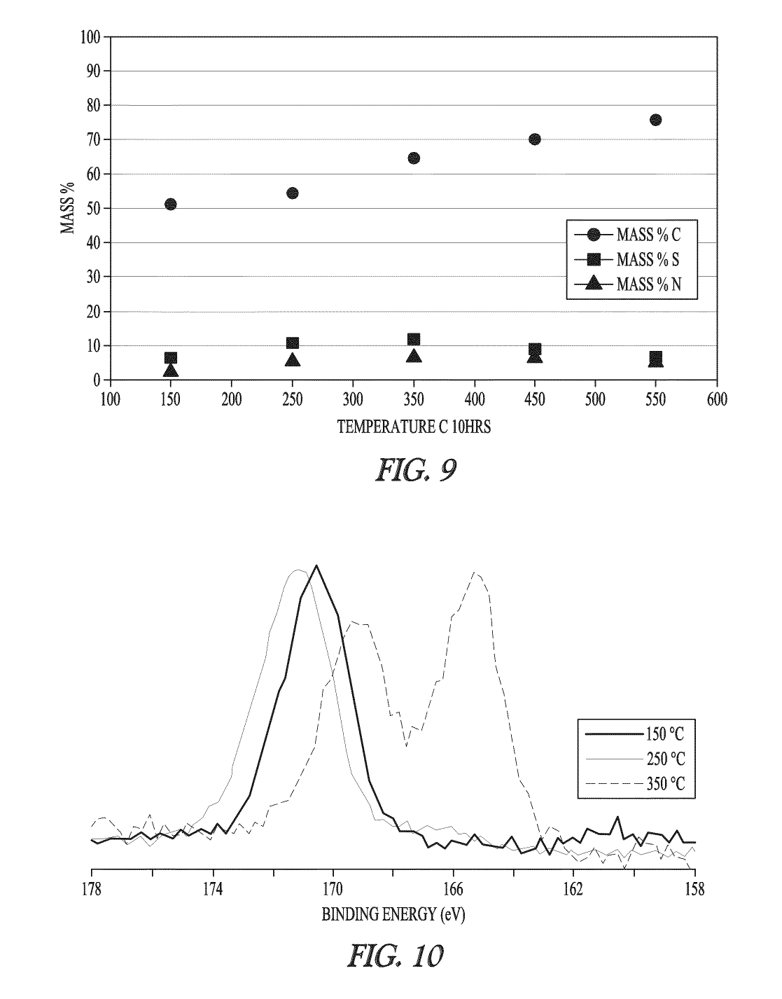

The ¹³C NMR spectrum of the material heated to 350° C. lacks the signals from the alkyl segment originating from taurine. This shows that the ethyl sulfonate groups break down when heated to 350° C., and that solid-state NMR is an excellent method for determining the optimum pyrolysis temperature. As the temperature is increased to 350° C. and beyond, the sulfur is reduced to a sulfone-type structure, and apparently incorporated into the aromatic scaffold. Elemental analysis shows that sulfur is retained in high percentages even after reaction at 550° C. (FIG. 9). However, sulfonic acid groups have likely been degraded to more reduced forms. This is deduced from an O:S atomic ratio <3, which does not provide enough oxygen for all sulfur to be in SO₃ groups. In addition, XPS suggests that many sulfonates have been reduced. Sulfonic groups bonded to alkyl carbons can be stable in water at 160° C. for over 24 h, while aromatically bonded sulfonic acids can break down under these conditions. Detailed characterization focused on the material treated at 250° C., which is the highest temperature that keeps the sulfonic groups intact. The empirical formula derived from CHNS analysis for GTaur materials produced with NaOH or KOH was determined to be C_{7.5}H_{6.75}NSO_{4.8}Na and C_{9.5}H_{13.75}N_{1.5}SO₆K, respectively. This compares to the precipitate prior to reaction of C₈H₁₃NSO₉Na. The observed loss of O and H is an indication of dehydration occurring. Interestingly, the GPyri material was highly dehydrated, at temperatures as low as 250° C. This material has an empirical formula of C_{27.8}H_{26.7}N_{4.7}O. FIG. 10 illustrates the sulfur 2p XPS spectra of GTaur materials prepared at three temperatures.

### Example 6

### Glucose-Phosphonate Material

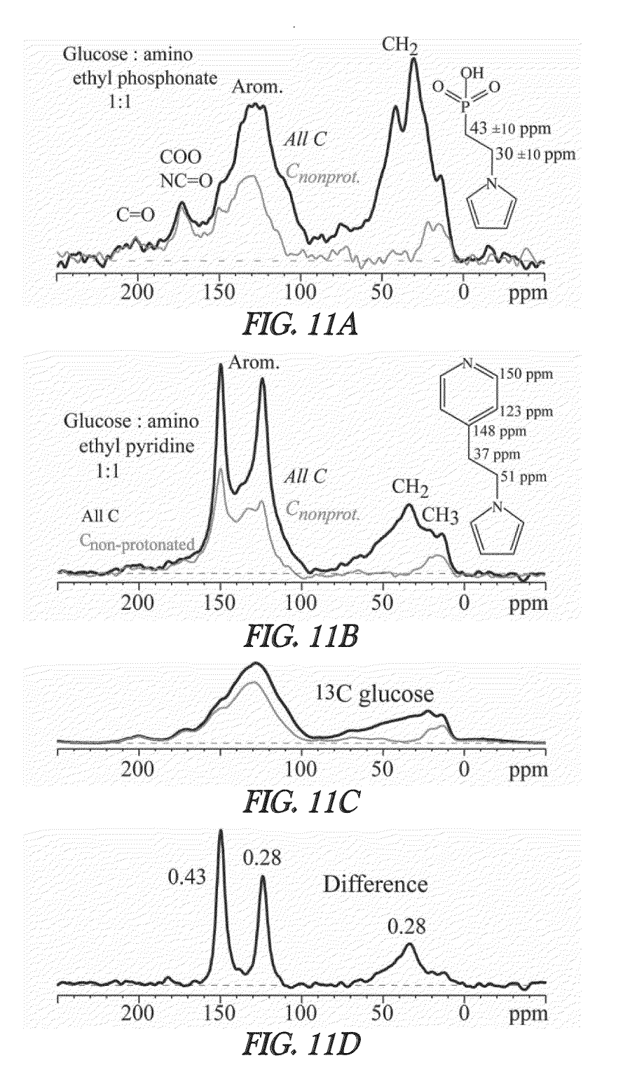

FIGS. 11a-d illustrate quantitative multiCP ¹³C NMR spectra of functionalized carbon-rich materials made by reaction of glucose with (a) aminoethyl phosphonic acid and (b-d) aminoethylpyridine, all in 1:1 molar ratios. Pyrolysis temperature: 250° C. No isotopic labeling was used. The spectrum of the phosphonate material in FIG. 11a generally shows the expected signals. The aromatic and CH₃ components closely resemble those of the sulfonate material. The prominent CH₂ signals are shifted to the right by about 10 ppm, in agreement with the chemical-shift predictions.

### Example 7

### Glucose-Pyridine Material

FIGS. 11b and c show ¹³C NMR spectra of GPyri without ¹³C enrichment and made with ¹³C₆ glucose, respectively. FIG. 11b shows distinctive peaks at 150 and 123 ppm, the characteristic chemical shifts of pyridine, which are absent in the spectrum of the glucose-derived matrix, FIG. 11c. This shows that pyridine is preserved as a pendent group in the structure. The difference spectrum in FIG. 11d, obtained with a scaling factor that minimizes the residual C═O and CH₃ signals, shows the amino ethyl pyridine CH₂ peaks between 35 and 60 ppm with the expected intensity.

Elemental analysis (Table 3) confirms that this material reacts in a fashion similar to the other analogs, since it contains twice as much nitrogen than the sulfonate or phosphonate materials due to the N in the pyridine ring. The nitrogen content (15% at 250° C. and 10% at 550° C.) is much higher than the values of 7.8%, 6.7%, or 4.1% reported for other carbon- and nitrogen-rich materials and is promising for platinum immobilization and other applications.

TABLE 3
Elemental compositions (in wt %) of GPyri and GTaur materials

| | C % | H % | N % | S % | Rem % |
|---|---|---|---|---|---|
| KOHGT250 | 35.9 | 4.8 | 6.7 | 10.1 | 42.5 |
| GNEP250 | 75 | 6.6 | 14.8 | 0 | 3.6 |
| GNPIP | 71 | 7.4 | 11.5 | 0 | 10.1 |

Unlike the SO₃ and PO₃ side groups, pyridine does not contain oxygen, and the oxygen content of the glucose-derived matrix can be estimated easily in the GPyri material. Elemental analysis shows little oxygen (<5% at 250° C.), consistent with NMR, where only 5% C═O, NC═O, or COO are detected. NMR has shown that the protons of the NH₂ group of the amine compound are mostly lost during the reaction; presumably, they combine with OH from glucose to form volatile H₂O and thus facilitate the loss of oxygen from the carbon-rich matrix.

### Example 8

### Piperidine-Functionalized Material

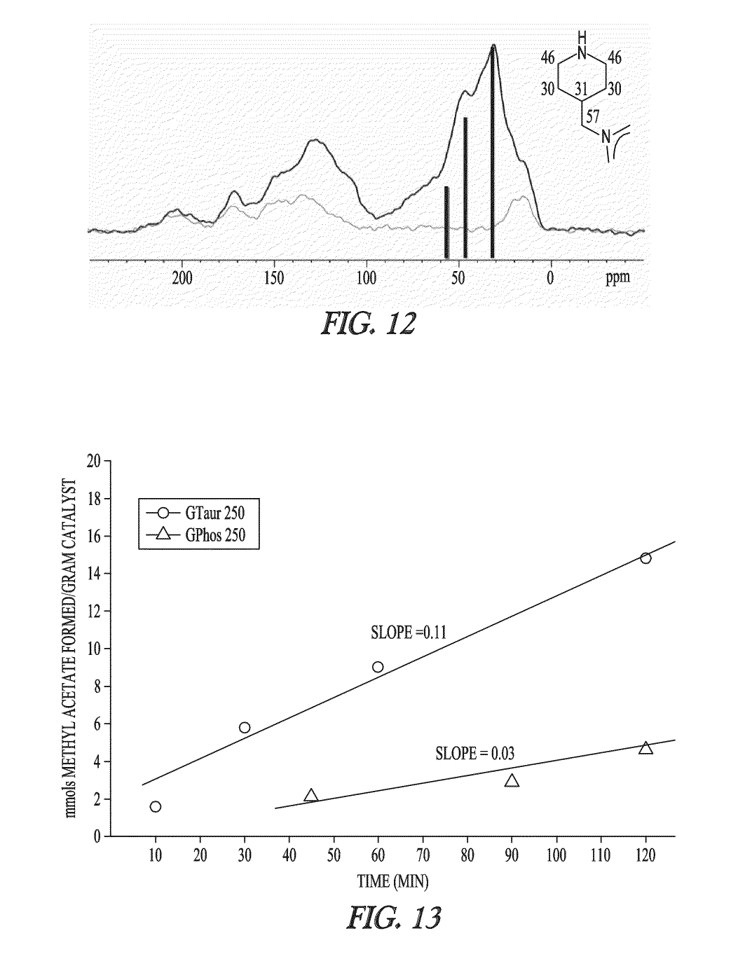

FIG. 12 illustrates multiCP ¹³C NMR spectra of amino-piperidine 250° C. analog of melanoidin reaction synthesis; black line: full spectrum; grey line: spectrum after dipolar dephasing. The ¹³C NMR spectra in FIG. 12 show that the synthesis works similarly well for another nitrogen-containing ring system, piperidine, which is a stronger base than pyridine, with pKa>10. The reaction works as expected with a methylene linker, and the dominant signals of the sp³-hybridized carbons on the piperidine ring have the expected 3:2:1 ratio. Additionally, elemental analysis shows a high level of N incorporation (see, Table 3).

### Example 9

### Catalytic Activity

Catalyst testing shows that the acid groups of GTaur and GPhos are active toward the esterification of methanol and acetic acid (FIG. 13, illustrating the mmoles of methyl acetate formed per gram of catalyst versus time). Although both activities are relatively low, they are substantially above the baseline conversion without catalyst present. GTaur is much more active than the GPhos materials, as expected based on its lower pKa value.

### Example 10

### Discussion of Examples 1-9

The materials had similar carbon-rich backbones resulting from carbon originating from glucose. The starting materials have a very high concentration of functional groups, and about two-thirds of these are retained intact after the thermal treatment at 250° C. Synthesis without a stoichiometric amount of base led to poorer taurine incorporation, with the NMR spectra showing sharp peaks from unreacted taurine. It seems that the use of NaOH to deprotonate the nitrogen was a successful approach for improving the reaction with the aldehyde on glucose and the uniformity of the material. The basic conditions and the increased temperature have enhanced the aromaticity compared to traditional Mailliard reaction insolubles.

The NMR analyses bring to light the complex nature of carbon materials made by pyrolysis and similar processes at low temperature, which is difficult to elucidate with IR, Raman, or surface techniques. Structural cartoons shown in numerous publications are not realistic models of carbon materials produced at moderate temperatures.

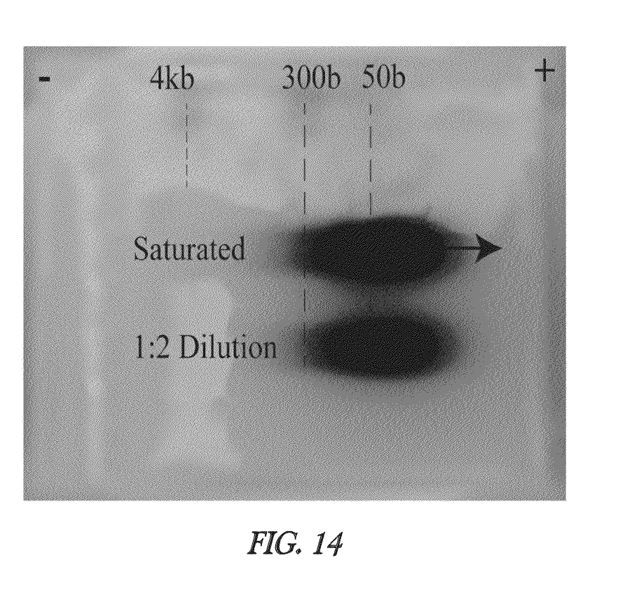

In addition to incorporation of numerous Brønsted acid sites, these materials display various interesting properties that can be tuned by changing the pyrolysis temperature, pyrolysis environment, and ratio of glucose:taurine. Materials which are produced at 250° C. with a 1:1:1 glucose:taurine:NaOH ratio form a colloid in water, but are insoluble in alcohols. This colloidal behavior in water makes it easy to disperse the material onto surfaces and template formation of nanocomposites. The polyanionic nature of the GTaur materials was verified by running them in a 1% agarose gel against a DNA ladder, as shown in FIG. 14. A migration rate similar to that of a 5 kDa fragment of DNA was observed. While the migration rate is a function of numerous factors including charge to mass, and morphology, this does indicate that the material is polyanionic and composed of relatively high molecular weight fragments. This could potentially be useful for formation of nanocomposites and deposition onto surfaces, recently shown by as an effective method to disperse Pd catalyst.

Given the robust nature of the reaction evident from the ever-growing library of molecules that react in an analogous manner, synthesis of multi-functional catalyst from this platform can utilize a variety of different sites. One excellent example of the utility of heterogeneous catalysts with both acidic and basic residues is the dehydration of glucose to hydroxyl-methyl furfural. Here, a base-catalyzed isomerization to the ketose sugars is followed by acid-catalyzed dehydration.

The aromatic materials produced have very high amounts of nitrogen incorporated into the aromatic matrix and remain stable even at fairly high pyrolysis temperatures. Such nitrogen-rich carbon materials have desirable properties as Pt supports in fuel cell applications, supercapacitors, and distribution of Pd catalyst.

The results indicate that the synthesis was successful and provides a simple one-step procedure to produce functionalized carbon materials through the reaction of a primary amine with a reducing sugar, which results in a high amount of nitrogen incorporation into the carbon scaffold with the moiety connected to nitrogen predominantly intact. This approach was successfully tested for several different primary amine analogs using numerous different moieties. The Maillard reaction platform synthesis introduced here has several desirable characteristics: First, this method can be easily modified to produce materials with numerous different functional groups incorporated by simply changing the R-group connected to the primary amine. Second, large amounts of nitrogen can be incorporated into the aromatic matrix. Third, charged side groups including sulfonate analogs are dispersed as a colloidal phase, which is can be used to synthesize thin films or nanocomposite materials. Lastly, given that glucose, a renewable material, is one of the primary reactants, these materials could be produced with renewable feedstocks, which could be an important consideration for large-scale applications such as energy storage with supercapacitors.

Part II. Engineering of Carbon Materials Produced from the Maillard Reaction Yields Hydrothermally Stable Sulfonic Acid Catalysts.

Summary: A hydrothermally stable sulfonic-acid catalyst with carbon-rich matrix has been synthesized and characterized. It was produced in a facile one-step reaction of glucose and taurine (H₃N—C₂H₄—SO₃) in a molar ratio near 4:1, combining the Maillard reaction and moderate pyrolysis at 250° C. The incorporation of alkyl-linked sulfonic-acid groups was verified by solid-state NMR. Repeated hydrothermal carbonization, for up to 96 h at 160° C., resulted only in moderate sulfur loss. To increase the surface area, the material was supported on SBA-15 and mesoporous carbon nanoparticles. On these two supports, the catalysts showed good esterification activity per sulfonate group.

General.

The sucrose, glucose, tetramethoxy silane, sulfuric acid, and taurine were purchased from Fisher and used without prior purification. The ¹³C uniformly labeled glucose was purchased from Cambridge Isotopes. Nitrogen (99.995%) was obtained from Airgas. The surfactant P123 was purchased from BASF.

### Example 11

### Bulk GT Synthesis

Mixtures of glucose taurine and base were co-precipitated with equal molar ratios of taurine and base (either sodium hydroxide or potassium hydroxide). The resulting material was added to two different molar ratios of additional glucose for greater backbone stability. The ratios were either 2:1 or 4:1 additional glucose to the original GT precipitate. The resulting solids were then pyrolyzed in a tube furnace under flowing argon. The materials were heated to 120° C. with a 1° C./min ramp and held for 2 hours to ensure completion of the Maillard reaction before continued heating to 250° C. with an additional dwell time of 10 hrs. Following pyrolysis, materials were ground into a fine powder using a cryomill with a 10 cps impact rate for three one-minute cycles. Following milling, materials were activated by soaking in 5 M KOH and heated for 4 hours at 150 C, and then washed with 5 M HCl followed by water until resulting liquid was no longer acidic. It was then dried overnight in an oven at 110° C. and used with no further modifications. These samples were labelled in accordance to the ratio of extra glucose to the 1:1 glucose taurine precipitate and which ion was used in their synthesis (e.g. GT2:1K or GT4:1Na).

### Example 12

### Synthesis of SBA-15

In a typical experiment, P123 (BASF, 7.0 g) was dissolved in 274 g of 1.6 M HCl and stirred for 1 h under 55° C. Then, 10.64 g of TMOS (tetramethoxy silane) was quickly added and the resultant solution was stirred for another 24 hours. A milky solution was obtained and transferred to an autoclave for additional 24 hours of hydrothermal treating under 120° C. After that, the solid was filtered and dried in air. To remove the surfactant, the SBA-15 material was calcined at 550° C. in air for 6 hours.

### Example 13

### Synthesis of GT on SBA-15

An aqueous solution with a 2:1 molar ratio of the glucose to taurine was added to the SBA-15. Then a sufficient amount of water was added to cover the SBA-15. It was placed in a hood to evaporate at room temperature. After evaporation, the catalyst was placed in a drying oven (110° C.) to initiate the Maillard reaction. After drying overnight in the drying oven, it was calcined in a tube furnace at 250° C. in nitrogen for 9⅓ hours. This material was used without further preparation. This sample is labeled “GT on SBA-15.”

### Example 14

### Mesoporous Carbon Nanoparticle (MCN) Synthesis

In a typical experiment, 4.0 g SBA-15 was mixed with 20 g of an aqueous solution containing 5.0 g of sucrose and 0.65 g 98% sulfuric acid. The resultant mixture was treated at 100° C. for 6 hours, followed by another 6 hours at 160° C. The process was repeated under the same condition but with 16 g of the same solution. Then the brownish solid was heated to 900° C. and held under nitrogen protection for 10 h. The silica was removed from the hybrids using 10% HF in ethanol/water mixture (50:50, v:v). After copious filtering and then drying the solid, the MCN material was obtained.

### Example 15

### Synthesis of the GT on MCN

The mesoporous carbon was functionalized in a way similar to the SBA-15. The mesoporous carbon was mixed with a glucose taurine solution in a 2:1 ratio. Then sufficient amount of water was added to cover the mesoporous carbon. The water was evaporated at room temperature and then the Maillard reaction was initiated by placing the reaction mixture in an oven at 110° C. overnight. Then the material was calcined at 250° C. in nitrogen for 9⅓ hours. This sample was labeled “GT on MCN.”

### Example 16

### Characterization

Surface area characterization was performed by physisorption using Kr as the adsorbing gas in a Micromeritics ASAP 2020. Elemental analysis results for the carbon, hydrogen, nitrogen, and sulfur (CHNS) were acquired using a PE 2100 Series II combustion analyzer (Perkin Elmer Inc., Waltham, Mass.). The chemical state of sulfur was analyzed using a Physical Electronics 550 Multitechnique XPS system employing a standard Al electron source. The samples were run at 10⁻⁹ Torr and mounted on double-sided tape. Charging was corrected for by adjusting the carbon peak to 284 eV. The number of sulfonic acid (R—SO₃H), carboxylic acid, and phenolic hydroxyl groups were measured using a titration method. One back-titration measured all the acidic functionalities, using a known amount of NaOH (˜0.1 g) dissolved in water with the catalytic material using sufficient time (˜2 hours) to exchange the sodium ions with all the hydrogen ions from the acid groups on the material. Then, a simple acid titration using sodium hydroxide was used to exchange the acidic protons from the sulfonic acid and carboxylic acid groups. It was assumed that the sulfonic acid, carboxylic acid, and hydroxyl groups were measured. The sulfonic acid groups were measured by the elemental analysis of the sulfur and the other groups were determined by subtraction of these measurements. The carbon materials were imaged with scanning electron microscopy (FEI Quanta-250 SEM), to investigate their morphologies and compare them with other sulfonated carbon catalysts. Surface area characterization was performed by physisorption using nitrogen as the adsorbing gas in a Micromeritics ASAP 2020. Analysis of the materials with a Perkin Elmer STA 6000 Simultaneous Thermal Analyzer (TGA), using a ramp rate of 10° C. in flowing air, was performed.

Esterification of acetic acid with methanol was used as the test reaction since it is sensitive to the number of acid sites and their pKₐ. The reactions were performed at 40° C. in an Alltech 10 mL reactor vial loaded (total volume 7 mL) with methanol (6 M) and acetic acid (3 M) in dioxane with the respective sulfonated carbon (10 mg). The reactions were run at low conversions (<5%) to allow comparison of initial rates. The reaction testing was performed at a range of stir rates so that it was determined that no mass transfer effects occurred as long as the stirring rate was above 200 rpm. The reaction product samples were analyzed on an Agilent 7890A GC equipped with a flame ionization detector. The sulfonated carbons were compared in terms of activity per mass of catalyst giving the units of mmol acetate formed min⁻¹ (g catalyst)⁻¹.

After full characterization of the fresh materials, the catalysts were subjected to three rounds of hydrothermal carbonization. The treatment subjected the catalyst material (0.5 g) to 160° C. DI water (7 mL) stirred in an Alltech reactor under autogenous pressure for 24 hours. The solid material was filtered and washed copiously (generally about 0.5 L of DI water) until the filtrate was clear and colorless. The hydrothermally treated sulfonated carbon was then characterized and subjected to two more hydrothermal carbonization treatments following the same protocol as the first treatment.

Solid State NMR. Quantitative solid state ¹³C NMR was applied at 100 MHz in a 9.4-T field with ¹H decoupling at 400 MHz, using a recently developed cross-polarization pulse sequence that utilized both a ramped ¹H CP power level and multiple cross polarization periods to allow for full ¹³C magnetization equilibration.

### Example 17

### NMR Characterization

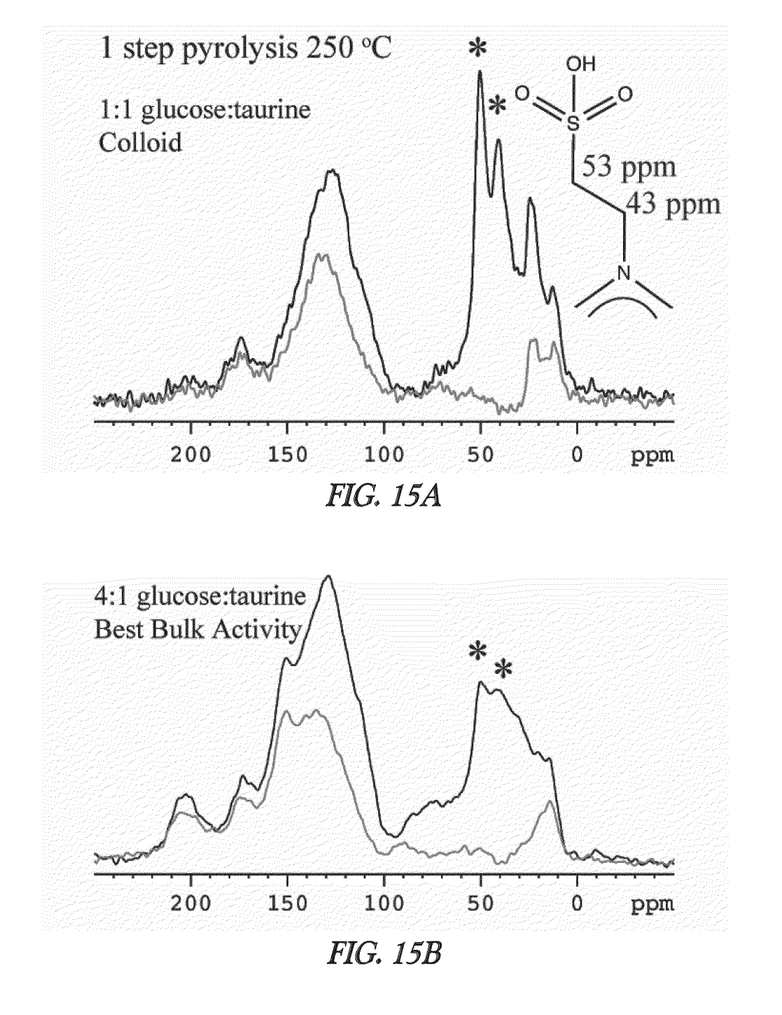

Solid-state ¹³C NMR was used to verify that NCH₂CH₂SO₃ groups characteristic of taurine are incorporated into the carbon backbone. FIGS. 15a-b illustrate a solid-state ¹³C NMR spectrum of sulfonic-acid functionalized GT4:1 material made from glucose and taurine. In FIG. 15a, the solid line is a quantitative multiCP spectrum, and the grey line is a multiCP spectrum after ¹H-¹³C dipolar dephasing, retaining only signals of nonprotonated C and mobile segments. FIG. 15b shows selective spectra of CH and CH₂ groups obtained by spectral editing. In the spectra shown in FIGS. 15a-b, signals of the characteristic CH₂ groups from taurine are clearly observed near 42 ppm (NCH₂) and 50 ppm (CH₂SO₃). Various combinations of glucose:taurine ratios were attempted, with addition of glucose to the colloid, followed by lyophilizing and pyrolysis; it was found that the characteristic CH₂ peaks of the taurine fragments were present under all conditions. FIGS. 15a-b show the increase in glucose corresponds to an increase in the peaks attributed to glucose carbon.

### Example 18

### Discussion of Examples 11-16

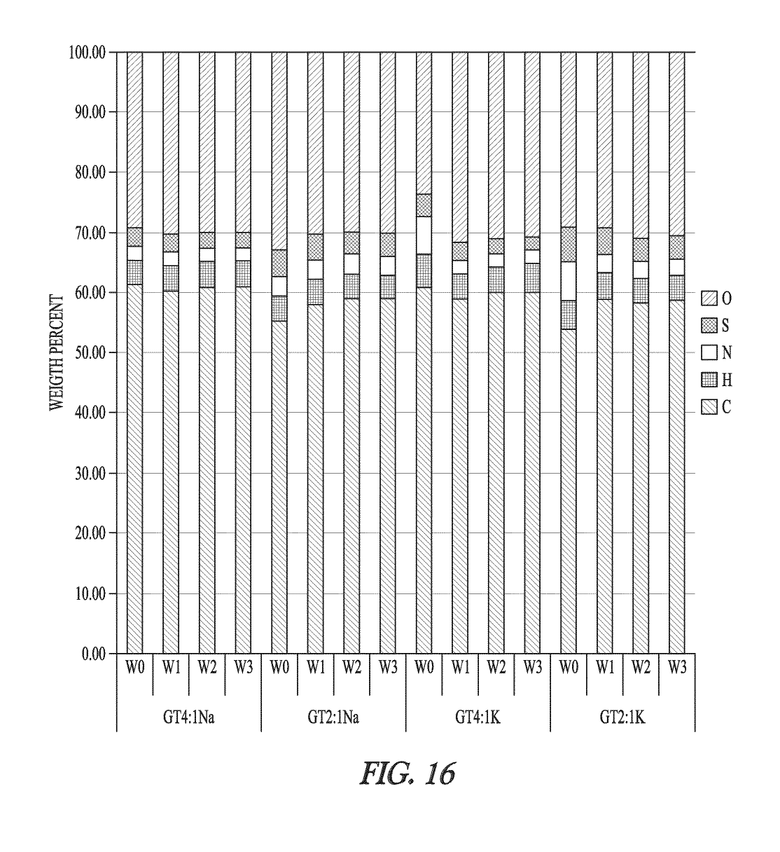

FIG. 16 illustrates the CHNS analysis of the bulk GT catalysts. There are four sets of four catalysts types that are labeled with either W0, indicating the initial samples, or W1-W3, indicating the number of hydrothermal carbonization treatments of each sample. The elemental compositions of the catalysts produced (see FIG. 16) include a significant amount of nitrogen from the amine group of taurine. Nitrogen and sulfur are in a 1:1 molar ratio as in the taurine starting material, which indicates that the linkage between nitrogen and sulfur was not lost during the synthesis. XPS data (not shown) confirmed the sulfur resided in the expected sulfonic acid with 168 eV binding energy in the Sp2 of sulfur. This material contains a significant amount of oxygen because of the moderate synthesis temperatures. The relatively low hydrogen fractions indicate significant aromaticity of the carbon support, which leads to the hydrothermal stability. In addition, the linkage between the carbon support and the active group must also be hydrothermally stable.

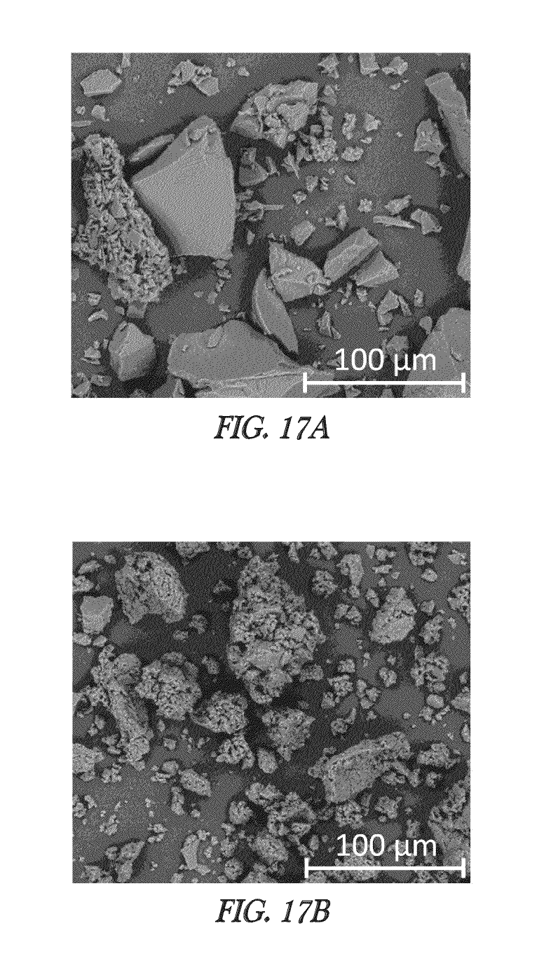

FIG. 17a-b illustrates SEM images of GTK (17a) and GTNa (17b). Both the GT2:1K and GT4:1K materials looked the same (e.g., the left image represents both). The BET data show that these materials have low surface area of <0.1 m²/g. In SEM images (see FIG. 17), these materials resemble compact shards of glass. From the analysis of the surface area alone, it can be deduced that many of the sulfonic-acid active groups are in the bulk. This was further verified by titrations of the better two catalysts showing that only 2-8% of the total sulfonic-acid groups are accessible for titration. This explains the observed low reaction rates and suggested that better dispersion of the sulfonic acid groups in a stable manner should improve catalytic performance. Therefore, GT was supported on SBA-15 and mesoporous carbon.

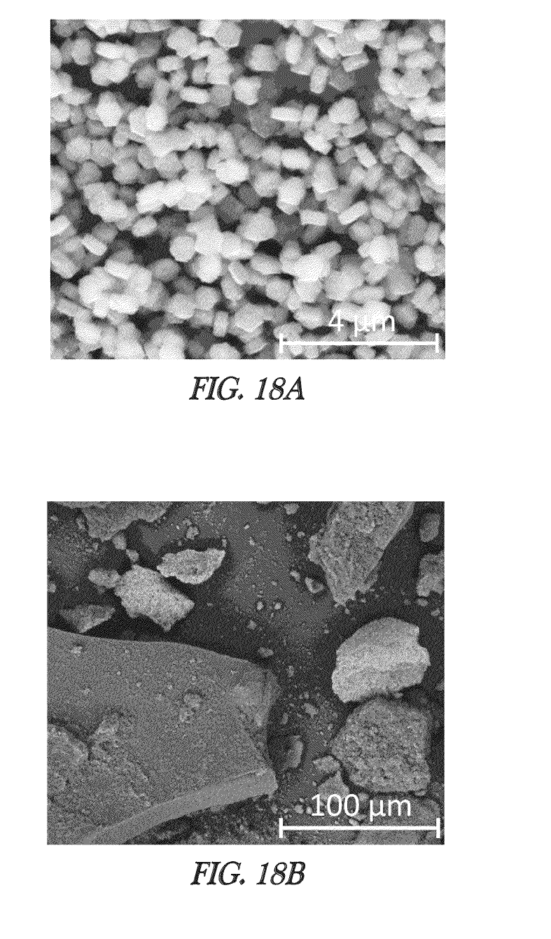

The surface area of these catalysts was significantly higher with the GT on SBA-15 catalyst having 314 m²/g and the GT on the MCN having 1110 m²/g. FIGS. 18a-b illustrate SEM images of GT on SBA-15 (18a) and GT on mesoporous carbon (18b). SEM images of the supported GT catalysts show some long-range order as expected from the synthesis method (see FIG. 18). The thermogravimetric analysis showed that the GT on the SBA-15 had about 6 wt % combustibles at 800° C. This corresponds well with the sulfur content, since the total combustibles times the expected weight percent of sulfur in the bulk GT2:1 samples gives close to the value measured by the elemental analysis.

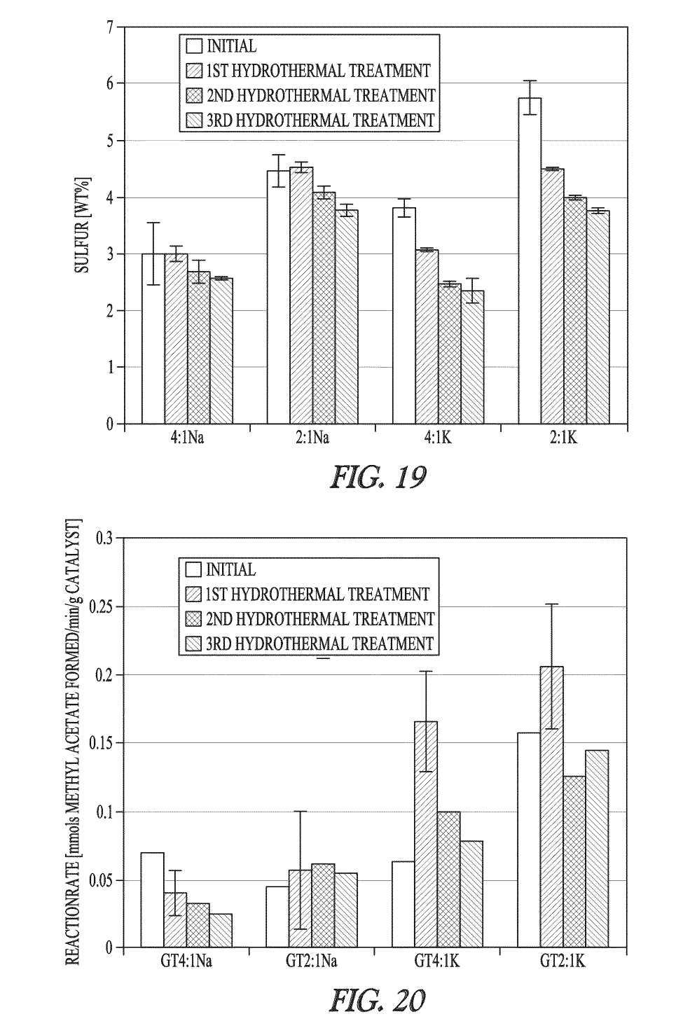

FIG. 19 illustrates sulfur retention of the GT catalysts after repeated hydrothermal carbonization treatments at 160° C. in liquid water for 24 hours. The materials lost only 20-40% of their sulfur (see FIG. 19), compared to 60-90% in other catalysts, which represents a great improvement over other materials. Nitrogen was lost to the same extent as the sulfur, indicating that the whole linker was lost and that the C—S bond does not limit the hydrothermal stability of the sulfonated carbon catalysts.

FIG. 20 illustrates the reaction rates for the catalysts throughout the hydrothermal carbonization treatments. The reaction rate data (see FIG. 20) shows an improvement in the stability of the reaction rates throughout the hydrothermal tests. While the data shows quite a bit of noise (a consequence of the low catalytic activity), it shows the relative consistency of the reaction rate even through the hydrothermal tests.

Table 4 illustrates details of the supported catalysts and the similar sulfur basis used for comparison of the supported catalysts. Since these materials more surface area could be helpful and it was presumed that the catalysts would have greater activity with better dispersion, supporting the GT catalysts on SBA-15 and mesoporous carbon was tested. As expected, when the GT colloid was supported on SBA-15 and mesoporous carbon, it did better than the bulk catalysts on a per sulfur basis. Comparing the amount of sulfur of the 2:1 catalysts and the amount of sulfur on the supported GT catalysts was the comparison basis for the catalysts. If the supported catalysts are compared on the same sulfur amount the GT2:1 catalysts have (average of 5 wt %) then they have a reaction rate of 0.72 mmols/min/(g catalyst) for the GT supported on SBA-15 and 0.85 mmols/min/(g catalyst) for the mesoporous carbon.

TABLE 4
Details of the supported catalysts and the similar sulfur basis used for
comparison of the supported catalysts.

| | | 2:1 GT on | 2:1 GT |
|---|---|---|---|
| | Units | SBA-15 | on MCN |
| Weight percent S | [% of the total | 0.35 | 0.24 |
| | mass] | | |
| Surface area | [m²/g] | 314 | 1110 |
| Similar sulfur basis | [−] | 14.3 | 20.8 |
| factor (5/weight | | | |
| percent %) | | | |
| Measured reaction rate | [mmols methyl | 0.041 | 0.051 |
| Reaction rate on a | acetate formed/ | 0.73 | 0.85 |
| similar sulfur basis | min/(g catalyst)] | | |

The clear improvement in reaction rates with greater surface area confirms the hypothesis of significant sulfur inclusion into the bulk of the catalyst where it is inaccessible to the reactants. With the titration data showing a similar fraction of 5% of the sulfonic acid groups as accessible, even the pyrolyzed catalysts' reaction rate would be similar as the supported catalysts if all of the sulfonic acid groups were accessible. Mesoporous carbon can be stable under these conditions and carbon layers on SBA-15 slow degradation in hot water; therefore, there is evidence that the supported catalysts are as stable as the bulk catalysts.

This material is an improvement over colloidal materials and with further addition of glucose, it can become a heterogeneous acid catalyst. This provides advantages in processability compared to a colloid as it is easier to separate and reuse than a colloid. This application of the Maillard reaction in a new way allows for a more hydrothermally stable attachment of the sulfonic acid group to be produced in a simple one-step reaction. Even with minimal optimization, it was shown that the new carbon catalysts retain sulfur better and have more consistent catalyst activity than similar catalysts in literature. Catalysts having high sulfur stability standpoints were made using sodium hydroxide. Catalysts having high activity were made from potassium hydroxide. Both had relatively low activities due to the embedded sulfonic acid groups that were inaccessible to the reactants. The surface area was increased by supporting the GT material on SBA-15 and mesoporous carbon nanoparticles, which both gave good results on a sulfur basis, and with both having sufficient hydrothermal stability for biorenewable processing. This method of creating a hydrothermally stable solid acid catalyst provides a pathway into a broad group of materials that can have applications in several fields that require hydrothermally stable supports and active groups.

### Part III. Hydrothermally Synthesized GT as a Solid-Acid Catalyst.

Summary: The Maillard reaction was successfully used to create hydrothermally stable carbon catalysts through pyrolysis synthesis. The Maillard reaction was used to create a new catalyst through a hydrothermal synthesis. The combination of glucose and taurine in a hydrothermal synthesis creates a solid that retains the sulfur, from the active group, even better than through pyrolysis synthesis. The synthesis temperatures ranged from 200-300° C. and it was found that the most stable catalysts were synthesized at 250° C. The catalytic activity seemed insensitive to differences in the changes of the glucose to taurine ratio from 1:1 to 2:1 at the 250° C. synthesis. At the 200° C. synthesis temperature, the activity is not stable through the hydrothermal testing and at the 300° C. synthesis temperature; the sulfur retention is not as stable as the catalysts synthesized at 250° C.

General. Glucose, 1,4 dioxane, glacial acetic acid, and methanol from Fisher Scientific. Taurine was purchased from Sigma-Aldrich. Glucose enriched with ¹³C was purchased from Cambridge Isotopes. All chemical were used with without further purification. Nitrogen gas (99.995%) was purchased from Airgas.

### Example 19

### Synthesis of Samples

The materials were prepared in batches based on 2 grams of glucose. The typical combination was 2 grams of glucose with 1.4 grams of taurine or about a 1:1 molar ratio of the two reactants. This was placed in a glass sleeve with 20 mL of DI water to solubilize the solution. This was placed in a stainless steel Parr reactor. The head space was pressurized with 500 psi of nitrogen and heated at 10° C./min until the desired temperature was reached. The solution was stirred at 200 RPM. It was held at the desired temperature for 18.5 hours then cooled to room temperature. The resulting solid was filtered, dried, and crushed to yield the final catalyst. For the material with a 2:1 molar ratio of glucose to taurine, the amount of taurine was halved to end up at the desired ratio.

The catalysts were designated via the nomenclature as follows: the ratio of the glucose to taurine mixture with the letters GT, the synthesis temperature, and then if it has been subjected to hydrothermal carbonization a W with a number of times (1-3). For example, sample 1:1GT250W2 is the 1:1 glucose to taurine ratio synthesized at 250° C. and it was subjected to two rounds of hydrothermal carbonization.

### Example 20

### Characterization

### Example 20a

### Catalyst Characterization

The carbon materials were imaged with scanning electron microscopy (FEI Quanta-250 SEM), to compare their morphologies with carbons generated by similar synthesis methods in the literature. Surface area characterization was performed by physisorption using nitrogen as the adsorbing gas in a Micromeritics ASAP 2020. Analysis of the materials with a Perkin Elmer STA 6000 Simultaneous Thermal Analyzer (TGA), using a ramp rate of 10° C. in flowing air was performed. Elemental analysis results for the carbon, hydrogen, nitrogen, and sulfur (CHNS) were acquired using a PE 2100 Series II combustion analyzer (Perkin Elmer Inc., Waltham, Mass.). The chemical state of sulfur was analyzed using a Physical Electronics 550 Multitechnique XPS system employing a standard Al electron source. The samples were run at 10⁻⁹ Torr and mounted on double-sided tape. Charging was corrected for by adjusting the carbon peak to 284 eV. Ion exchange titrations were run to measure the number of acidic groups present on the sulfonated carbons. The number of sulfonic acid (R—SO₃H), carboxylic acid, and phenolic hydroxyl groups were measured using titration. One back titration measured all the acidic functionalities. It used a known amount of NaOH (˜0.1 g) dissolved in water with the catalytic material using sufficient time (˜2 hours) to exchange the sodium ions with all the hydrogen ions from the acid groups on the material. Then, a simple acid titration using sodium hydroxide was used to exchange the acidic protons from the sulfonic acid and carboxylic acid groups. It was assumed that the sulfonic acid, carboxylic acid, and hydroxyl groups were measured. The sulfonic acid groups were measured by the elemental analysis of the sulfur and the other groups were determined by subtraction of these measurements.

### Example 20b

### TPD Characterization

Temperature-programmed desorption was carried out in a home-built setup equipped with a gas chromatograph (Varian CP-4900 Micro-GC) and a mass spectrometer (Pfeiffer Omnistar) for on-line product analysis. Typically, 20 mg of sample were loaded into a fixed-bed quartz reactor, which was placed in a self-constructed furnace with an isothermal zone of 4 cm at the upper temperature limit. Weakly adsorbed water was effectively removed within 1 h at 373 K in a He stream passed at a flow rate of 25 mL/min. Experiments were started thereafter by linearly heating the reactor at 5 K/min to 1323 K. This temperature was maintained for 30 min before cooling to room temperature. The basis for the samples is compared as a per carbon mass of the remaining sample after the experiment.

### Example 20c

### Reaction Testing

Esterification of acetic acid with methanol was used as the test reaction since it is sensitive to the number of acid sites and their pKₐ. The reactions were performed at 40° C. in an Alltech 10 mL reactor vial loaded (total volume 7 mL) with methanol (6 M) and acetic acid (3 M) in dioxane with the respective sulfonated carbon (10 mg). The reactions were run at low conversions (<5%) to allow comparison of initial rates. The reaction testing was performed at a range of stir rates so that it was determined that no mass transfer effects occurred as long as the stirring rate was above 200 rpm. The reaction product samples were analyzed on an Agilent 7890A GC equipped with a flame ionization detector. The sulfonated carbons were compared in terms of activity per mass of catalyst giving the units of mmol acetate formed min⁻¹ (g catalyst)⁻¹.

After full characterization of the fresh materials, the catalysts were subjected to three individual hydrothermal carbonization treatments. Each treatment placed the catalyst material (0.5 g), stirred in DI water (7 mL) at 160° C., in the Alltech reactors under autogenous pressure for 24 hours. The solid material was filtered and washed extensively (generally about 0.5 L of DI water) until the filtrate was clear and colorless. The hydrothermally treated sulfonated carbon was then characterized and subjected to two more hydrothermal carbonization treatments following the same protocol as the first treatment.

### Example 21

### Discussion and Characterization of Examples 19-20

### Example 21a

### General

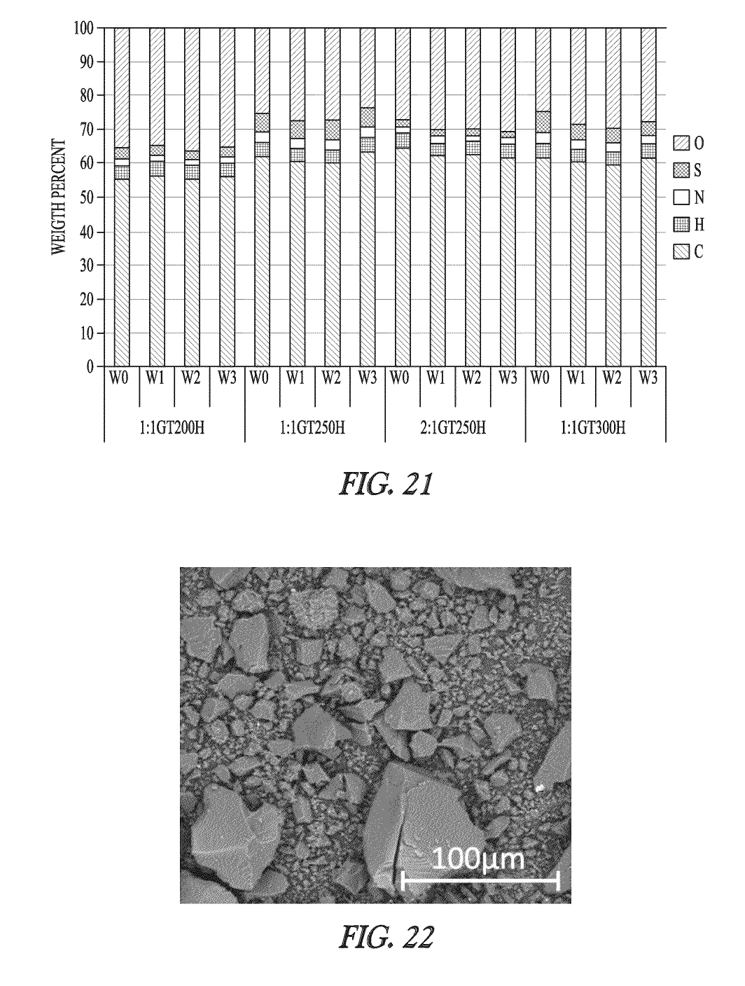

FIG. 21 illustrates elemental analysis data of the catalysts. There are four sets of four included with the W0 data points representing the initial samples, and the W1-W3 represent the number of hydrothermal tests the sample was subjected to. The materials made by this method seem to have minimal changes throughout the hydrothermal testings from the elemental analysis (see FIG. 21). The 200° C. synthesis has the least amount of carbon while the three other synthesis have similar weight percent of carbon. The sulfur and the nitrogen are in a one to one ratio as expected from the Maillard reaction. This indicated the linker was able to be retained during synthesis. Increasing the glucose to taurine ratio from 1:1 to 2:1 decreased the amount of sulfur by little over a half. Unexpectedly, the 200° C. material also had low weight percent of sulfur. As the sulfur was removed from the solid, so too was the nitrogen, evidencing the strength of the linker on the nitrogen bond. The material is also relatively hydrogen poor which indicates aromaticity. It is this aromaticity that gives the carbon framework hydrothermal stability while the sulfonic acid must be attached via aliphatic linkages to ensure its hydrothermal stability.

The BET data showed all these catalysts had <0.1 m²/g. From this data, sulfur must be incorporated into the bulk of the catalyst. Titrations were done and showed that only ˜10% of the sulfur groups were accessible via titration. This means these catalysts would enjoy better reaction rates if they were better dispersed. FIG. 22 illustrates a SEM of the 1:1GT200 sample. The SEM data (see FIG. 22) shows these catalysts to look more like glass shard-particles from incompletely pyrolyzed glucose rather than spheres from hydrothermally formed glucose. Only one SEM picture was included because all the other catalysts formed similar type particles.

### Example 21b

### Temperature-Programmed Desorption

Profiles of the volatile compounds detected in the course of TPD experiments contain valuable information about the structural state of a carbonaceous compound, the extent of functionalization, and the thermal stability of heteroatomic species present in the material. Since the carbonyl group in glucose reacts with the amino group in taurine via the Maillard reaction, it can be expected that sulfonic acid entities are attached to the hydrothermally formed carbon via N-substituted alkyl chains. In view of this, SO₂ and NH₃ were monitored in addition to CO, CO₂, CH₄, H₂ and H₂O. Quantitative information gives a deeper insight into the thermally induced processes taking place. For this reason all aforementioned compounds, excepting hydrogen and water, were calibrated and analysed by means of gas chromatography.

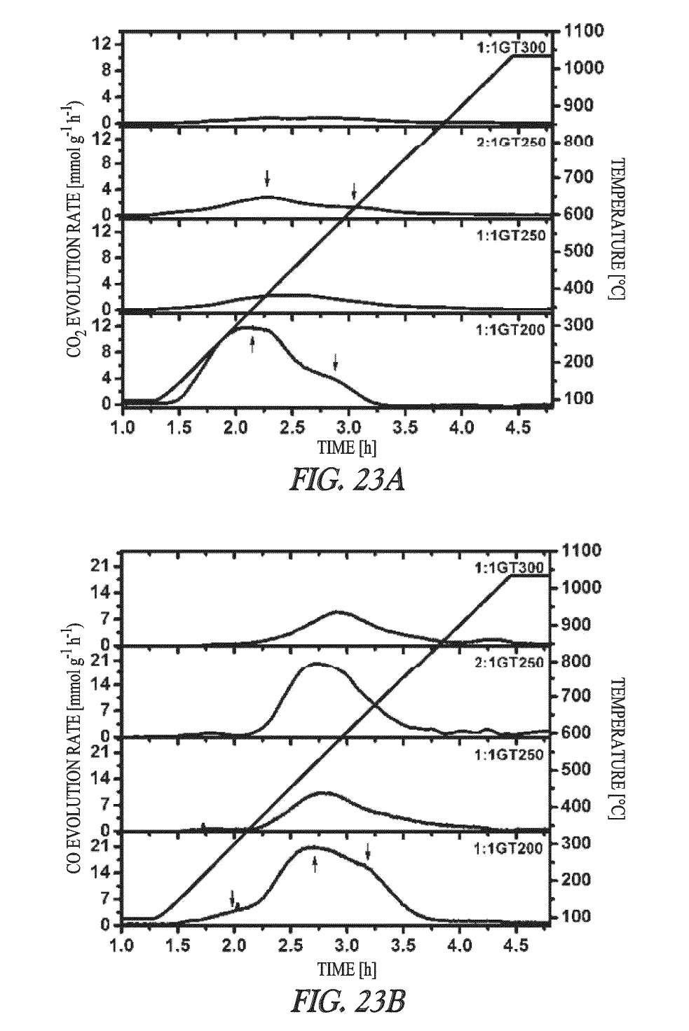

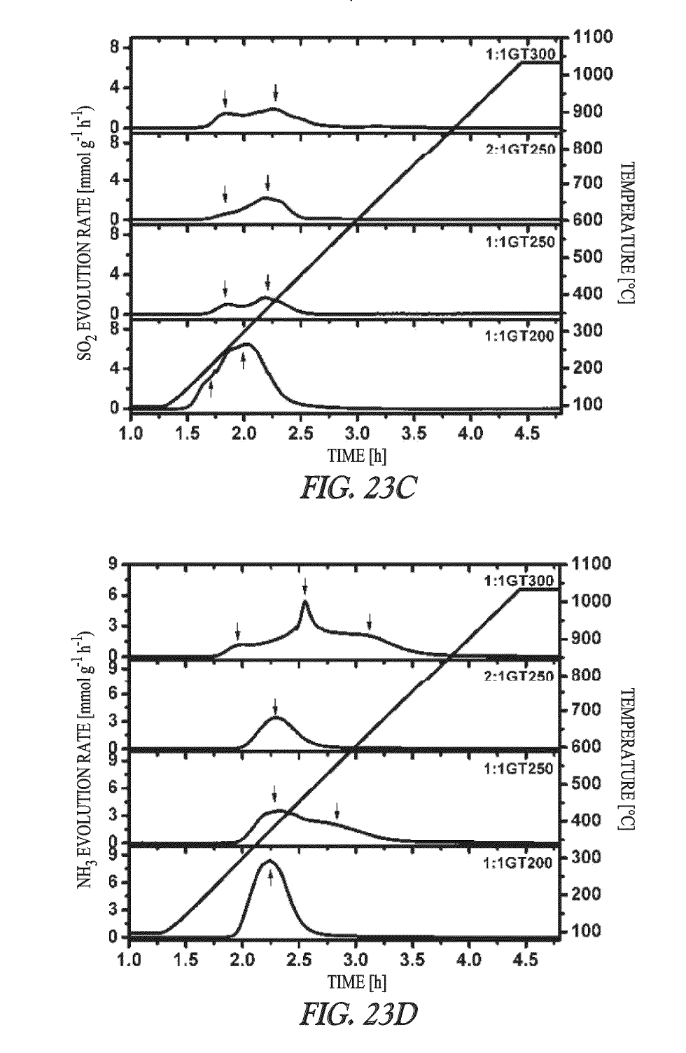

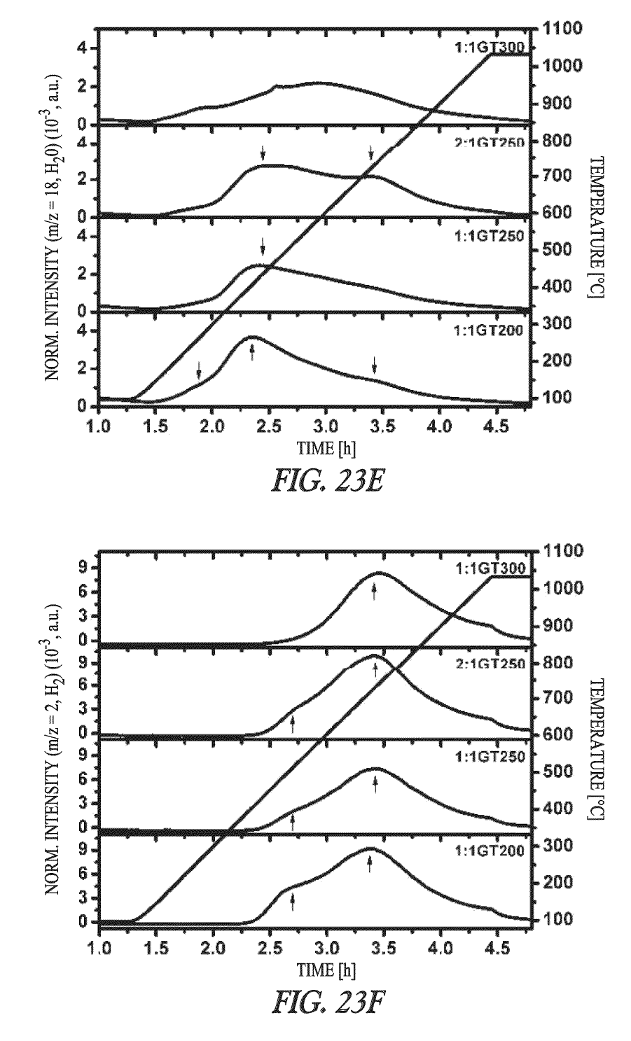

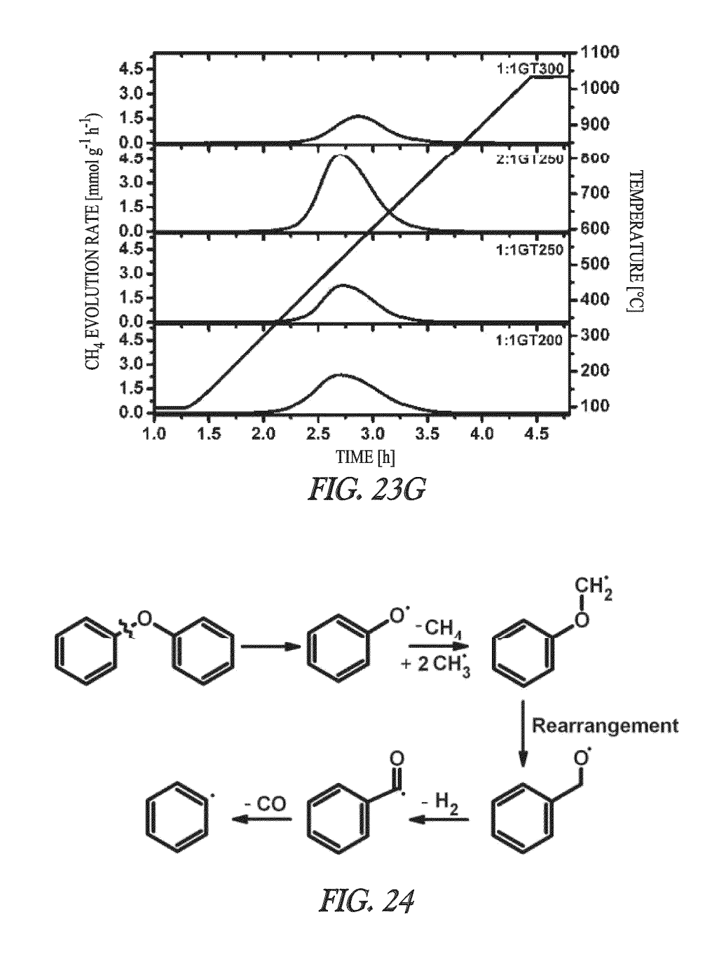

FIGS. 23a-g illustrate the evolution of (a) CO₂, (b) CO, (c) SO₂, (d) NH₃, (e) H₂O, (f) H₂, and (g) CH₄ during TPD analysis of aminoethanesulfonic acid containing hydrothermal carbons. FIG. 23a shows the evolution of CO₂ of the hydrothermally prepared carbons. Three characteristic regions are observed in 1:1GT200. The broad region between 150° C. and 460° C. is an overlap of the contributions arising from the decomposition of carboxylic acids (˜260° C.) and anhydrides (˜380° C.). The most thermally stable species are lactones, whose presence is reflected by the shoulder centred at 560° C. As observed by the CO₂ evolution of 1:1GT250, increasing the synthesis temperature to 250° C. leads to almost a complete disappearance of the carboxylic acid feature and a significant reduction of the anhydride/lactone functionalities. According to the calculated amounts summarized in Table 1, 70% less CO₂ evolves from 1:1GT250 compared to 1:1GT200. An increase in the synthesis temperature from 250° C. to 300° C. decreases the CO₂ contribution by around 60%. This is associated with the gradual increase of condensation between the building blocks of the carbonaceous structure as further evidenced by the steady decrease in CH₄ formation (FIG. 23g) from initially 1698 to finally 1003 iumol/g. Methane is linked to the thermal decomposition of aliphatic, hydroaromatic, and aryl methyl groups. As expected from the nature of glucose, which is used as the carbon precursor, less CH₄ evolves with increasing aromatic character of the carbon. In addition, the methane peak maximum is shifted by 60° C. to 570K for 1:1GT300, whereas the remaining samples have this feature located at 510° C. Interestingly, the amount of CO₂ detected during the TPD of 2:1GT250 is comparable to 1:1GT250. Evidently, doubling the molar ratio of glucose to taurine has no significant effect on the CO₂ evolution. This agrees with the elemental analysis data which saw little increase in the amount C between the two samples. However, the amount of methane behaves in the same manner as the molar ratio changes, indicating that this volatile compound is in fact linked to the carbon precursor.

TABLE 1
Specific molar concentrations of CO, CO₂, CH₄, SO₂ and NH₃ derived
from TPD experiments.

| | | CO | CO₂ | CH₄ | SO₂ | NH₃ | |
|---|---|---|---|---|---|---|---|
| Entry | Sample | [μmol · g_{C}⁻¹] | [μmol · g_{C}⁻¹] | [μmol · g_{C}⁻¹] | [μmol · g_{C}⁻¹] | [μmol · g_{C}⁻¹] | n_{SO2}:n_{NH3} |
| 16978 | 1:1GT200 | 24250 | 11484 | 1698 | 3548 | 3356 | 1.06 |
| 16979 | 1:1GT250 | 10469 | 3496 | 1306 | 1676 | 2854 | 0.59 |
| 16980 | 2:1GT250 | 18710 | 3580 | 2734 | 1206 | 1437 | 0.84 |
| 16981 | 1:1GT300 | 8613 | 1445 | 1003 | 1888 | 3433 | 0.55 |

As can be observed in FIG. 23a, CO profiles exhibit a broad feature with a peak maximum located at around 550° C. Oxygenated species present in carbon nanostructures decomposing to CO are anhydrides, phenols, ethers, and carbonyl groups. In view of their structural and binding state differences, these functionalities are thermally decomposed over a wide temperature range. However, the building units of hydrothermally prepared carbons are prone to undergo further condensation reactions, is evidenced by the formation of CO. A plausible pathway as depicted in FIG. 24 for this temperature regime involves the cleavage of diaryl ethers yielding phenoxy radicals that upon reacting with methyl radicals and subsequent methane elimination lead to methoxyphenyl radicals. After rearrangement and dehydrogenation, a benzene radical is formed via CO elimination. Growth of the aromatic system can subsequently proceed by recombination of benzene radicals. CO amounts calculated from the TPD profiles drastically drop from 24250 to 8613 μmol/g_{carbon}, for the carbons synthesized with a glucose-to-taurine ratio of 1:1. Since the thermal decomposition of oxygen functional groups involved in the formation of carbon monoxide begins at values beyond the highest applied synthesis temperature of 300° C., the determined inversely proportional trend between CO evolution and synthesis temperature indicates that a significant amount of CO arises from the carbonization processes, reflecting different degrees of condensation between 1:1GT200, 1:1GT250 and 1:1GT300. Hence, merely a small fraction of CO is associated with the thermal depletion of 0-containing surface species. These findings are in line with the CO amounts obtained for 1:1GT250 and 2:1GT250. Doubling the glucose-to-taurine ratio increases the amount of released CO from 10469 to 18710 μmol/g_{carbon}, which corresponds to a rise of about 80%.

According to FIG. 24, H₂ is formed prior to the decarbonylation step, which is in line with the lower temperature onset of hydrogen evolution in comparison to that of the CO formation. FIG. 23f displays a shoulder at 550° C. that has been attributed to this process. The main phenomenon represented by the peak located at around 750° C. is related to the H₂ evolution as a consequence of polyaromatic condensation reactions taking place in various superimposed steps. This leads to the formation of large polycondensed units that represent the main building blocks of the pyrolyzed carbon. No dehydrogenation is observed above 900° C., which is related to the homolytical cleavage of C—H bonds. From this it can be inferred that most likely only a negligible fraction of sites are saturated after heat treatment.

Evolution of H₂O is related to various processes. This is reflected in FIG. 23e by the presence of several peaks. Weakly adsorbed water has no contribution since the samples were dried at 100° C. for 1 h prior to the TPD experiments. The shoulder observed below 300° C. can be attributed not only to the dehydration of adjacent carboxylic acid species yielding cyclic anhydrides, but also to condensation of neighbouring sulfonic acid entities. Glucose as the carbon precursor consists of five OH groups distributed over six carbon atoms. For this reason the hydrothermal carbons are expected to contain a large number of phenolic species. The thermal decomposition of OH groups can lead to the formation of pyrolysis water over a broad temperature range clearly evidenced by the H₂O evolution between 400° C. and 800° C.

In addition to the changes observed to which the hydrothermal carbons are subjected in the course of the TPD experiments, the success of functionalization is evidenced by the evolution of sulphur dioxide (FIG. 23c) and ammonia (FIG. 23d). Sulfonic acid entities decompose to SO₂ at temperatures below 200° C., whereas the onset of NH₃ evolution is nearly 300° C. This finding implies that the thermal stability of the linker between the carbon backbone and the SO₃H groups is significantly more than the acid entities themselves. Furthermore, less sulfonic acid groups are attached to the hydrothermal carbon when increasing the synthesis temperature from 200° C. to 250° C. This is evidenced by the reduction of SO₂ from 3548 to 1676 μmol/g_{carbon}. Interestingly, the effect is reverted when the temperature is further increased to 300° C. Ammonia behaves in a similar manner. However, more ammonia evolves from sample 1:1GT300 than 1:1GT200, meaning that 300° C. is beneficial for the Maillard reaction, but at the same time detrimental for the attached sulfonic acid groups. Increasing the glucose-to-taurine ratio from 1:1 to 2:1 leads to a lower specific molar concentration of sulfonic acid groups. As evidenced by the arrows in FIG. 23c, all the samples exhibit the presence of two or even three types of sulfonic acid groups differing in terms of thermal stability. A more heterogeneous character is observed in the case of ammonia, since 1:1GT200 and 2:1GT250 show one broad peak centred below 400° C., whereas 1:1GT250 releases ammonia in two different stages with the second maximum located below 600° C. Three different steps are observed in the case of 1:1GT300. The calculated SO₂-to-NH₃ molar ratio of 1:1GT200 corresponds to the expected nitrogen and sulphur molarity found in taurine. All other samples exhibited values below 1. A reason for this might be the degradation of sulfonic acid groups under hydrothermal conditions. While the linker remains attached to the carbonaceous framework, the acid group is decomposed. Furthermore, NH₃ may be formed in the course of the hydrothermal synthesis via deamination of taurine. Under these conditions, ammonia is capable of reacting with oxygen functional groups available in the carbon compound leading to nitrogen species that decompose to ammonia during the TPD experiments. To a certain extent, both processes could overlap, enhancing the deamination route with increasing temperature.

### Example 21c

### ATR-FTIR

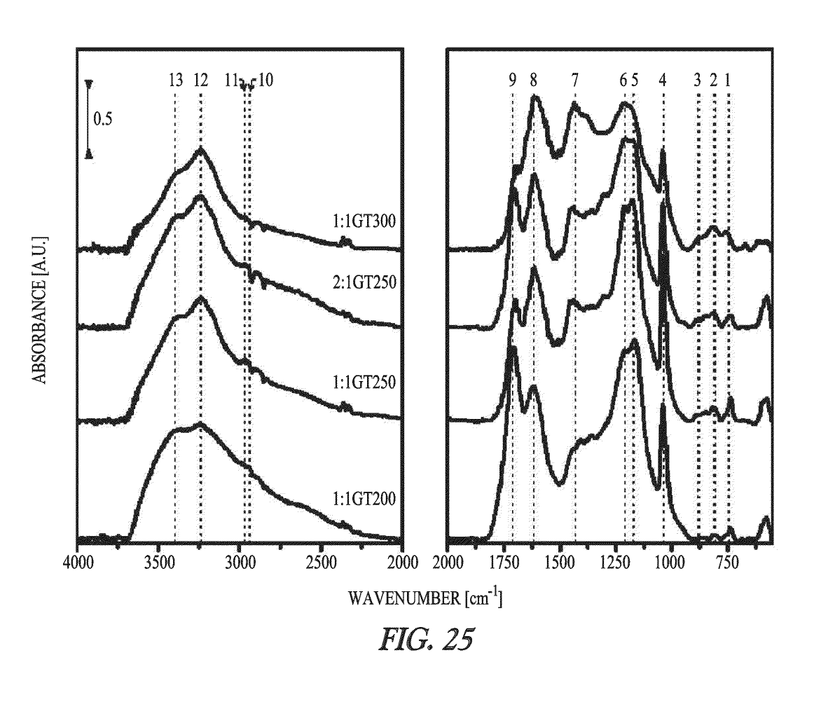

Spectroscopic methods are suitable for monitoring the hydrothermal transformation of glucose into carbonaceous products and the concomitant modification with sulfonic acid entities via the Maillard reaction. FIG. 25 illustrates the ATR-FTIR spectra of the obtained hydrothermal carbons. The presence of aromatic domains is confirmed by the band located at 1614 cm⁻¹ (8), which corresponds to the C═C stretching vibrations of aromatic and furanic rings. This is further evidenced by the feature at 1436 cm⁻¹ (7), attributed to C—C stretching vibrations of aromatic rings. Aromatic C—H out-of-plane bending vibrations encountered in the region between 900 and 700 cm⁻¹ (1-3) underpin the aromatic character of the samples. As expected from the structure of glucose and from the results derived from the TPD experiments, phenolic species exist on the carbon compounds as suggested by the broad band at 3400 cm⁻¹ (13). The presence of further oxygen functional groups can be confirmed by the feature at 1703 cm⁻¹ (9), which are ascribable to C═O vibrations of carbonyl, quinone, ester, or carboxyl moieties. Differences in terms of carbonization degree can be encountered among 1:1GT200, 1:1GT250, and 1:1GT300. The relative intensity between the C═O (9) and the aromatic C═C stretching vibrations (8) clearly decreases with increasing synthesis temperature, suggesting a higher extent of condensation between the building blocks constituting the carbonaceous compounds accompanied by a loss of oxygen. These observations are in line with the changes observed for CH₄ and CO during TPD. Sulfonic acid groups give rise to the bands at 1209 cm⁻¹ (6) and 1039 cm⁻¹ (4) that can be respectively attributed to the antisymmetric and symmetric SO₃ stretching vibrations. Bands at 3241 cm⁻¹ (12) and 1174 cm⁻¹ (5) indicate the presence of nitrogen species as expected from the employment of taurine during hydrothermal synthesis. The former is ascribable to N—H stretching vibrations of aliphatic secondary amines, whereas the latter may be assigned to C—N stretching vibrations of secondary amines. The carbon materials also possess aliphatic structures as evidenced by the bands in the region between 3000 and 2900 cm⁻¹ (10, 11), which arise from the stretching vibrations of aliphatic C—H bonds. These findings suggest the covalent binding of the sulfonic acid entities to the carbon backbone through an alkyl linker, underpinning the success of the Maillard reaction for functionalization purposes.

### Example 21d

### Solid-State NMR

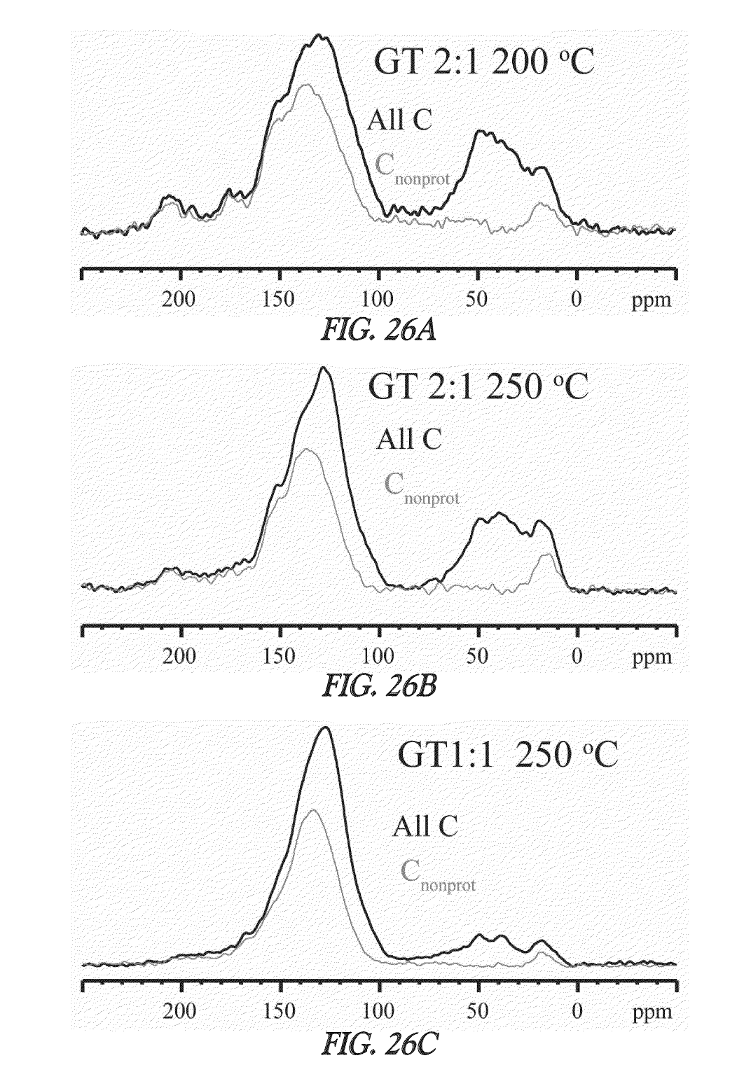

Solid state ¹³C NMR of the 2:1GT250 (FIGS. 26a-b), shows the presence of strong methylene peaks which are consistent with what would be expected from this Maillard reaction synthesis. FIG. 26c illustrates the solid state ¹³C NMR of the 1:1GT250. Additionally, many of the spectral features present are rigorously characterized GT materials produced in dry pyrolysis. It is very likely that the aromatic groups are underrepresenting with respect to the methylene groups given that this spectra was collected using standard cross polarization. The dipolar dephased spectra which removed most of the signal of carbons that have strong ¹H-¹³C dipolar coupling, shows that the resonance between 40-60 ppm is likely CH and CH₂'s and the peaks below 30 ppm are predominantly CH₃'s, which is consistent with levulinic and acetic acid derived from glucose breakdown. Further, little dephasing in the aromatic region is consistent with the aromatic units being heavily substituted and therefore crosslinked. The aromatic section of the spectra (150-100 ppm) looks very similar to the typical hydrothermal material with the qualifier of the overrepresentation of the carbon attached to hydrogen. Although the strong levulinic acid ketone and CH₃ peaks observed with hydrochar produced from carbohydrate-only feedstocks are not obversed, there is indication that the reactions that glucose is undergoing have been significantly altered compared to glucose-derived hydrocharbon.

### Example 21e

### Sulfur Retention and Reaction Rates

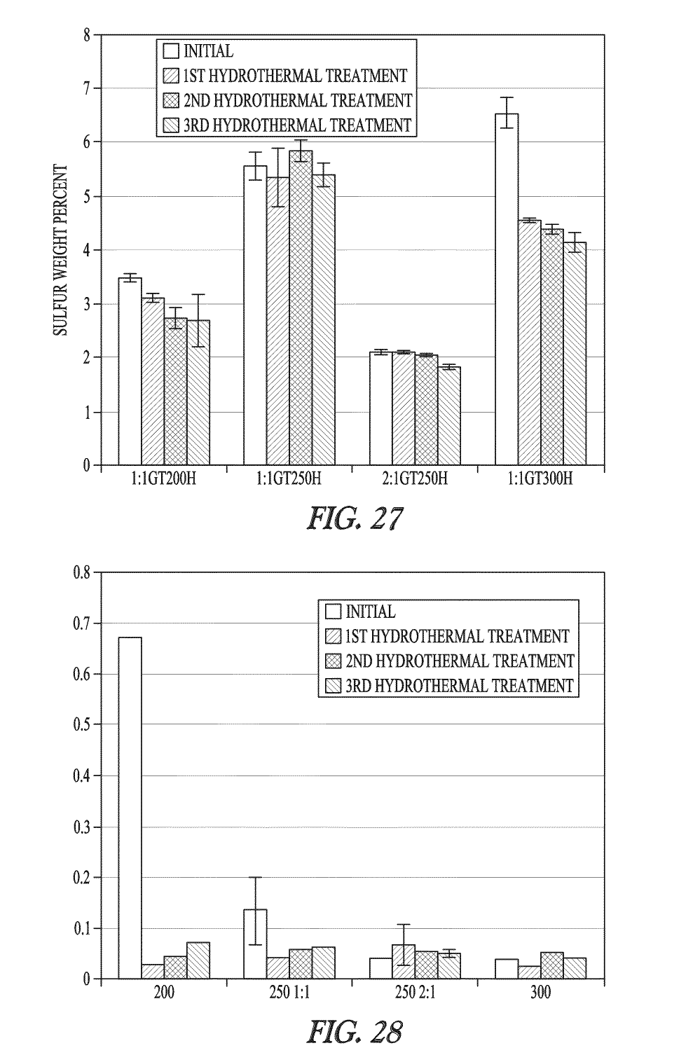

FIG. 27 illustrates sulfur retention data for the hydrothermal GT catalysts. Each hydrothermal test was 24 hours in 160° C. liquid water. FIG. 28 illustrates reaction rate data for the hydrothermal GT catalysts throughout the hydrothermal carbonization treatments. This synthesis method created the most stable catalysts yet of the glucose taurine materials. The sulfur loss of the 1:1GT300H was the most significant at 36% while the 1:1GT200H lost 23% and the 2:1GT250H lost 12%. The 1:1GT250H was the best at retaining the sulfur throughout the hydrothermal carbonization treatments as it had no significant loss of sulfur. These catalysts are excellent at retaining their sulfur.

The reaction rate data tells the story of stability for the materials synthesized above 200° C. As a result of the low activity of most of the catalysts, the data are noisy. In those materials, the differences in the activity are insignificant throughout each of the hydrothermal tests. There were no significant reaction differences in the 1:1GT250 sample and the 2:1GT250 sample. As seen from elemental analysis, the sulfur was a little over twice as concentrated in the sample not enriched in glucose but this did not significantly affect the reaction rates. Supporting this material on high surface area material promotes better reaction rates which also could be used for this material as well.

### Example 21f

### Summary

These materials represent an additional improvement in the glucose taurine catalysts. They have excellent retention of their initial sulfur loading. The 1:1GT250 retained its sulfur the best with only an insignificant amount of loss. The reaction rates are stable throughout the hydrothermal carbonization treatments provided that the synthesis temperature is high enough. Extensive characterization showed the material to be a successful use of the Maillard reaction to create a sulfonic acid attached with an alkyl linker through a nitrogen atom. The characterization also showed higher temperatures increased the incorporation of the nitrogen atom into the matrix at the cost of sulfur incorporation. This suggests a maximum effective synthesis temperature to incorporate both the linker and the sulfonic acid. The Maillard reaction, as studied for this catalyst synthesis, is an effective way to incorporate the linkages needed for a hydrothermally stable acid catalyst.

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present invention. Thus, it should be understood that although the present invention has been specifically disclosed by specific embodiments and optional features, modification and variation of the concepts herein disclosed may be resorted to by those of ordinary skill in the art, and that such modifications and variations are considered to be within the scope of embodiments of the present invention.

### Additional Embodiments

The following exemplary embodiments are provided, the numbering of which is not to be construed as designating levels of importance:

Embodiment 1 provides a functionalized carbon matrix having the structure:

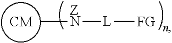

wherein
wherein at each occurrence Z is independently selected from H and a bond to CM,
CM is a substituted or unsubstituted carbon matrix,
at each occurrence linker L is independently substituted or unsubstituted (C₁-C₄₀)hydrocarbyl interrupted by 0, 1, 2, or 3 atoms chosen from —S—, —O—, and —NR³—,
at each occurrence functional group FG is independently chosen from —OR³, —OOR³, —OC(O)N(R³)₂, —CN, —CF₃, —OCF₃, —C(O), methylenedioxy, ethylenedioxy, —N(R³)₂, —SR³, —SOR³, —SO₂N(R³)₂, —C(O)R³, —C(O)C(O)R³, —C(O)CH₂C(O)R³, —C(S)R³, —C(O)OR³, —OC(O)R³, —C(O)N(R³)₂, —OC(O)N(R³)₂, —C(S)N(R³)₂, —N(R³)C(O)R³, —N(R³)N(R³)₂, —N(R³)N(R³)C(O)R³, —N(R³)N(R³)C(O)OR³, —N(R³)N(R³)CON(R³)₂, —N(R³)SO₂R³, —N(R³)SO₂N(R³)₂, —N(R³)C(O)OR³, —N(R³)C(O)R³, —N(R³)C(S)R³, —N(R³)C(O)N(R³)₂, —N(R³)C(S)N(R³)₂, —N(COR³)COR³, —N(OR³)R³, —C(═NH)N(R³)₂, —C(O)N(OR³)R³, —C(═NOR³)R³, —S(O)(OR¹)₃, —S(O)(OR¹)₂R², —S(O)(OR¹)R²₂, —S(O)R²₂, —OS(O)(OR¹)₃, —OS(O)(OR¹)₂R², —OS(O)(OR¹)R²₂, —OS(O)R²₂, —S(O)₂OR¹, —S(O)₂R², —OS(O)₂OR¹, —OS(O)₂R², —P(O)(OR¹)₂, —P(O)(OR¹)R², —P(O)R²₂, —OP(O)(OR¹)₂, —OP(O)(OR¹)R², —OP(O)R²₂, and a substituted or unsubstituted nitrogen-containing (C₁-C₂₀)heterocycle, wherein
   at each occurrence R¹ is independently chosen from —H, a counterion, and substituted or unsubstituted (C₁-C₂₀)hydrocarbyl,
   at each occurrence R² is independently chosen from substituted or unsubstituted (C₁-C₂₀)hydrocarbyl, and
   at each occurrence R³ is independently chosen from —H and substituted or unsubstituted (C₁-C₂₀)hydrocarbyl, and
degree of substitution n is about 1 to about 1,000,000.

Embodiment 2 provides the functionalized carbon matrix of Embodiment 1, wherein the functionalized carbon matrix is a reaction product of at least one of hydrothermal carbonization and pyrolysis of a reducing sugar and an amine having the structure H₂N-L-FG.

Embodiment 3 provides the functionalized carbon matrix of any one of Embodiments 1-2, wherein the carbon matrix is a reaction product of at least one of a hydrothermal carbonization and pyrolysis of a reducing sugar and the amino-group of an amine having the structure H₂N-L-FG.

Embodiment 4 provides the functionalized carbon matrix of any one of Embodiments 1-3, wherein Z is a bond to CM.

Embodiment 5 provides the functionalized carbon matrix of any one of Embodiments 1-4, wherein at each occurrence L is independently chosen from substituted or unsubstituted (C₁-C₄₀)hydrocarbyl.

Embodiment 6 provides the functionalized carbon matrix of any one of Embodiments 1-5, wherein at each occurrence L is independently chosen from (C₁-C₁₀)hydrocarbyl.

Embodiment 7 provides the functionalized carbon matrix of any one of Embodiments 1-6, wherein at each occurrence L is independently chosen from methylene, ethylene, propylene, butylene, pentylene, hexylene, heptylene, octylene, nonylene, decylene, and phenylene.

Embodiment 8 provides the functionalized carbon matrix of any one of Embodiments 1-7, wherein at each occurrence L is independently chosen from methylene, ethylene, butylene, and phenylene.

Embodiment 9 provides the functionalized carbon matrix of any one of Embodiments 1-8, wherein at each occurrence FG is independently chosen from an acidic functionality or a salt thereof.

Embodiment 10 provides the functionalized carbon matrix of any one of Embodiments 1-9, wherein at each occurrence FG is independently chosen from —S(O)(OR¹)₃, —S(O)(OR¹)₂R², —S(O)(OR¹)R²₂, —S(O)R²₂, —OS(O)(OR¹)₃, —OS(O)(OR¹)₂R², —OS(O)(OR¹)R²₂, —OS(O)R²₂, —S(O)₂OR¹, —S(O)₂R², —OS(O)₂OR¹, —OS(O)₂R², —P(O)(OR¹)₂, —P(O)(OR¹)R², —P(O)R²₂, —OP(O)(OR¹)₂, —OP(O)(OR¹)R², and —OP(O)R²₂.

Embodiment 11 provides the functionalized carbon matrix of any one of Embodiments 1-10, wherein at each occurrence FG is independently chosen from S(O)₂OR¹ and P(O)(OR¹)₂.

Embodiment 12 provides the functionalized carbon matrix of any one of Embodiments 1-11, wherein at each occurrence FG is independently chosen from S(O)₂OH and P(O)(OH)₂.

Embodiment 13 provides the functionalized carbon matrix of any one of Embodiments 1-12, wherein at each occurrence FG is independently chosen from S(O)₂OH and P(O)(OH)₂.

Embodiment 14 provides the functionalized carbon matrix of any one of Embodiments 1-13, wherein at each occurrence FG is independently chosen from a basic functionality.

Embodiment 15 provides the functionalized carbon matrix of any one of Embodiments 1-14, wherein at each occurrence FG is independently chosen from a substituted or unsubstituted nitrogen-containing (C₁-C₂₀)heterocycle.

Embodiment 16 provides the functionalized carbon matrix of any one of Embodiments 1-15, wherein at each occurrence FG is independently chosen from a substituted or unsubstituted nitrogen-containing monocyclic (C₁—O₅)heterocycle.

Embodiment 17 provides the functionalized carbon matrix of any one of Embodiments 1-16, wherein at each occurrence FG is independently chosen from substituted or unsubstituted pyridinyl and substituted or unsubstituted piperidinyl.

Embodiment 18 provides the functionalized carbon matrix of any one of Embodiments 1-17, wherein at each occurrence FG is independently chosen from pyridin-4-yl and piperidin-4-yl.

Embodiment 19 provides the functionalized carbon matrix of any one of Embodiments 1-18, wherein n is at least 2 and the functionalized carbon matrix comprises at least one FG that is an acidic functionality or a salt thereof and at least one FG that is a basic functionality.

Embodiment 20 provides the functionalized carbon matrix of any one of Embodiments 1-19, wherein at each occurrence L is independently chosen from ethylene, butylene, methylene, and phenylene, and at each occurrence FG is independently chosen from S(O)₂OH, P(O)(OH)₂, pyridin-4-yl, and piperidin-4-yl.

Embodiment 21 provides a composition comprising the functionalized carbon material of any one of Embodiments 1-20.

Embodiment 22 provides the composition of Embodiment 21, wherein the composition comprises a colloidal composition.

Embodiment 23 provides a compound comprising: the functionalized carbon matrix of any one of Embodiments 1-22; and at least one metal cation coordinated thereto.

Embodiment 24 provides the compound of Embodiment 23, wherein the metal cation is chosen from at least one of Cu⁺, Cu²⁺, Fe²⁺, Fe³⁺, Pb²⁺, Pb⁴⁺, Sn²⁺, Sn⁴⁺, Ni²⁺, Ni³⁺, Hg₂²⁺, Hg²⁺, Cr²⁺, Cr³⁺, Mn²⁺, Mn³⁺, Co²⁺, Co³⁺, Ag⁺, Zn²⁺, Cd²⁺, Sc³⁺, Na⁺, K⁺, Mg²⁺, Pd²⁺, Pt²⁺, Li⁺, Al³⁺, Cs⁺, V²⁺, V⁴⁺, Ru³⁺, Ru⁴⁺, Rh³⁺, Ce³⁺, and Ca²⁺.

Embodiment 25 provides the compound of any one of Embodiments 23-24, wherein about 10 wt % to about 80 wt % of the compound is the metal cation.

Embodiment 26 provides the compound of any one of Embodiments 23-25, wherein about 30 wt % to about 60 wt % of the compound is the metal cation.

Embodiment 27 provides a nanomaterial comprising the functionalized carbon matrix of any one of Embodiments 1-26.

Embodiment 28 provides the nanomaterial of Embodiment 27, wherein the nanomaterial comprises a nanocomposite.

Embodiment 29 provides the nanocomposite of Embodiment 28, wherein the nanocomposite comprises ferro-magnetic particles.

Embodiment 30 provides the nanocomposite of any one of Embodiments 28-29, wherein about 10 wt % to about 80 wt % of the nanocomposite is metal.

Embodiment 31 provides the nanomaterial of any one of Embodiments 27-30, wherein the nanomaterial has a particle size of about 0.1 nm to about 200 nm.

Embodiment 32 provides the nanomaterial of any one of Embodiments 27-31, wherein the nanomaterial has a particle size of about 5 nm to about 10 nm.

Embodiment 33 provides a composite material, comprising a high surface area substrate; the functionalized carbon matrix of any one of Embodiments 1-32 deposited thereon.

Embodiment 34 provides the composite material of Embodiment 33, wherein the high surface area substrate comprises mesoporous silica.

Embodiment 35 provides the composite material of any one of Embodiments 33-34, wherein the high surface area substrate comprises about 100 m²/g to about 5000 m²/g.

Embodiment 36 provides the composite material of any one of Embodiments 33-35, wherein the high surface area substrate comprises about 300 m²/g to about 2000 m²/g.

Embodiment 37 provides the composite material of any one of Embodiments 33-36, wherein the functionalized carbon matrix comprises nanoparticles.

Embodiment 38 provides the composite material of any one of Embodiments 33-37, wherein the functionalized carbon matrix comprises a film on the mesoporous silica.

Embodiment 39 provides the composite material of any one of Embodiments 33-38, wherein the composite material comprises a catalyst.

Embodiment 40 provides the composite material of Embodiment 39, wherein the composite material is resistant to hydrothermal degradation.

Embodiment 41 provides a functionalized carbon matrix having the structure:

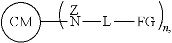

wherein
at each occurrence Z is independently selected from H and a bond to CM,
CM is a substituted or unsubstituted carbon matrix,
at each occurrence L is chosen from ethylene, butylene, methylene, and phenylene,
at each occurrence FG is chosen from S(O)₂OH, P(O)(OH)₂, pyridin-4-yl, and piperidin-4-yl, and
degree of substitution n is about 1 to about 1,000,000.

Embodiment 42 provides a method of making a functionalized carbon matrix, the method comprising

obtaining or providing a composition comprising
a reducing sugar, and
a functionalized amine having the structure H₂N-L-FG, wherein
   at each occurrence linker L is independently substituted or unsubstituted (C₁-C₄₀)hydrocarbyl interrupted by 0, 1, 2, or 3 atoms chosen from —S—, —O—, and —NR³—,
   at each occurrence functional group FG is independently chosen from —OR³, —OOR³, —OC(O)N(R³)₂, —CN, —CF₃, —OCF₃, —C(O), methylenedioxy, ethylenedioxy, —N(R³)₂, —SR³, —SOR³, —SO₂N(R³)₂, —C(O)R³, —C(O)C(O)R³, —C(O)CH₂C(O)R³, —C(S)R³, —C(O)OR³, —OC(O)R³, —C(O)N(R³)₂, —OC(O)N(R³)₂, —C(S)N(R³)₂, —N(R³)C(O)R³, —N(R³)N(R³)₂, —N(R³)N(R³)C(O)R³, —N(R³)N(R³)C(O)OR³, —N(R³)N(R³)CON(R³)₂, —N(R³)SO₂R³, —N(R³)SO₂N(R³)₂, —N(R³)C(O)OR³, —N(R³)C(O)R³, —N(R³)C(S)R³, —N(R³)C(O)N(R³)₂, —N(R³)C(S)N(R³)₂, —N(COR³)COR³, —N(OR³)R³, —C(═NH)N(R³)₂, —C(O)N(OR³)R³, —C(═NOR³)R³, —S(O)(OR¹)₃, —S(O)(OR¹)₂R², —S(O)(OR¹)R²₂, —S(O)R²₂, —OS(O)(OR¹)₃, —OS(O)(OR¹)₂R², —OS(O)(OR¹)R²₂, —OS(O)R²₂, —S(O)₂OR¹, —S(O)₂R², —OS(O)₂OR¹, —OS(O)₂R², —P(O)(OR¹)₂, —P(O)(OR¹)R², —P(O)R²₂, —OP(O)(OR¹)₂, —OP(O)(OR¹)R², —OP(O)R²₂, and a substituted or unsubstituted nitrogen-containing (C₁-C₂₀)heterocycle, wherein
      at each occurrence R¹ is independently chosen from —H, a counterion, and substituted or unsubstituted (C₁-C₂₀)hydrocarbyl,
      at each occurrence R² is independently chosen from substituted or unsubstituted (C₁-C₂₀)hydrocarbyl, and
      at each occurrence R³ is independently chosen from —H and substituted or unsubstituted (C₁-C₂₀)hydrocarbyl; and

subjecting the composition to at least one of pyrolysis and hydrothermal carbonization, to provide a functionalized carbon matrix having the structure:

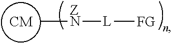

wherein at each occurrence Z is independently selected from H and a bond to CM, CM is a substituted or unsubstituted carbon matrix, and degree of substitution n is about 1 to about 1,000,000.

Embodiment 43 provides the method of Embodiment 42, wherein the reducing sugar is at least one chosen from glucose, fructose, glyceraldehyde, galactose, lactose, maltose, erythrose, threose, ribose, arabinose, xylose, lyxose, allose, altrose, gulose, idose, talose, ribulose, xylulose, psicose, sorbose, tagatose, and cellobiose.

Embodiment 44 provides the method of any one of Embodiments 42-43, wherein the reducing sugar is glucose.

Embodiment 45 provides the method of any one of Embodiments 42-44, wherein at each occurrence L is independently chosen from ethylene, butylene, methylene, and phenylene.

Embodiment 46 provides the method of any one of Embodiments 42-45, wherein at each occurrence FG is independently chosen from S(O)₂OH, P(O)(OH)₂, pyridin-4-yl, and piperidin-4-yl.

Embodiment 47 provides the method of any one of Embodiments 42-46, wherein the functionalized amine is chosen from aminoethyl sulfonic acid, aminoethyl phosphonic acid, 4-(2-aminoethyl)pyridine, and 4-aminomethylpiperidine.

Embodiment 48 provides the method of any one of Embodiments 42-47, wherein the pyrolysis comprises treating with a temperature of about 200° C. to about 700° C.

Embodiment 49 provides the method of any one of Embodiments 42-48, wherein the pyrolysis comprises treating with a temperature of about 240° C. to about 600° C.

Embodiment 50 provides the method of any one of Embodiments 42-49, wherein the hydrothermal carbonization comprises treating with a temperature of about 80° C. to about 350° C.

Embodiment 51 provides the method of any one of Embodiments 42-50, wherein the hydrothermal carbonization comprises treating with a temperature of about 90° C. to about 150° C.

Embodiment 52 provides the method of any one of Embodiments 42-51, wherein the hydrothermal carbonization comprises treating with a temperature of about 160° C. to about 220° C.

Embodiment 53 provides the method of any one of Embodiments 42-52, wherein the method further comprises treating with a temperature of about 30° C. to about 120° C. for about 30 minutes to about 14 days prior to the hydrothermal treatment or pyrolysis.

Embodiment 54 provides the method of any one of Embodiments 42-53, wherein the hydrothermal carbonization or pyrolysis occurs for a duration of about 1 minute to about 7 days

Embodiment 55 provides the method of any one of Embodiments 42-54, wherein the composition further comprises a base.

Embodiment 56 provides the method of any one of Embodiments 42-55, further comprising making a colloidal mixture comprising the functionalized carbon matrix and a fluid.

Embodiment 57 provides the method of any one of Embodiments 42-56, further comprising coordinating at least one metal cation to the functionalized carbon matrix.

Embodiment 58 provides the method of any one of Embodiments 42-57, further comprising templating a nanocomposite with the functionalized carbon matrix.

Embodiment 59 provides a functionalized carbon material made by the method of any one of Embodiments 42-58.

Embodiment 60 provides a method of making a functionalized carbon matrix, the method comprising

obtaining or providing a composition comprising
glucose, and
a functionalized amine chosen from aminoethyl sulfonic acid, aminoethyl phosphonic acid, 4-(2-aminoethyl)pyridine and 4-aminomethylpiperidine; and

subjecting the composition to at least one of pyrolysis and hydrothermal carbonization, to provide a functionalized carbon matrix having the structure:

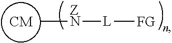

wherein
wherein at each occurrence Z is independently selected from H and a bond to CM,
CM is a substituted or unsubstituted carbon matrix,
at each occurrence L is independently chosen from methylene, ethylene, butylene, and phenylene,
at each occurrence FG is independently chosen from S(O)₂OH, P(O)(OH)₂, pyridin-4-yl, and piperidin-4-yl, and
degree of substitution n is about 1 to about 1,000,000.

Embodiment 61 provides a method of making a nitrogen-rich carbon material, the method comprising:

obtaining or providing a composition comprising
a reducing sugar, and
a functionalized amine having the structure H₂N-L-FG, wherein
   at each occurrence linker L is independently substituted or unsubstituted (C₁-C₄₀)hydrocarbyl interrupted by 0, 1, 2, or 3 atoms chosen from —S—, —O—, and —NR³—,
   at each occurrence functional group FG is independently chosen from —OR³, —OOR³, —OC(O)N(R³)₂, —CN, —CF₃, —OCF₃, —C(O), methylenedioxy, ethylenedioxy, —N(R³)₂, —SR³, —SOR³, —SO₂N(R³)₂, —C(O)R³, —C(O)C(O)R³, —C(O)CH₂C(O)R³, —C(S)R³, —C(O)OR³, —OC(O)R³, —C(O)N(R³)₂, —OC(O)N(R³)₂, —C(S)N(R³)₂, —N(R³)C(O)R³, —N(R³)N(R³)₂, —N(R³)N(R³)C(O)R³, —N(R³)N(R³)C(O)OR³, —N(R³)N(R³)CON(R³)₂, —N(R³)SO₂R³, —N(R³)SO₂N(R³)₂, —N(R³)C(O)OR³, —N(R³)C(O)R³, —N(R³)C(S)R³, —N(R³)C(O)N(R³)₂, —N(R³)C(S)N(R³)₂, —N(COR³)COR³, —N(OR³)R³, —C(═NH)N(R³)₂, —C(O)N(OR³)R³, —C(═NOR³)R³, —S(O)(OR¹)₃, —S(O)(OR¹)₂R², —S(O)(OR¹)R²₂, —S(O)R²₂, —OS(O)(OR¹)₃, —OS(O)(OR¹)₂R², —OS(O)(OR¹)R²₂, —OS(O)R²₂, —S(O)₂OR¹, —S(O)₂R², —OS(O)₂OR¹, —OS(O)₂R², —P(O)(OR¹)₂, —P(O)(OR¹)R², —P(O)R²₂, —OP(O)(OR′)₂, —OP(O)(OR¹)R², —OP(O)R²₂, and a substituted or unsubstituted nitrogen-containing (C₁-C₂₀)heterocycle, wherein
      at each occurrence R¹ is independently chosen from —H, a counterion, and substituted or unsubstituted (C₁-C₂₀)hydrocarbyl,
      at each occurrence R² is independently chosen from substituted or unsubstituted (C₁-C₂₀)hydrocarbyl, and
      at each occurrence R³ is independently chosen from —H and substituted or unsubstituted (C₁-C₂₀)hydrocarbyl; and

subjecting the composition to pyrolysis comprising a temperature of about 500° C. to about 2000° C., to provide a nitrogen-rich carbon material.

Embodiment 62 provides the method of Embodiment 61, wherein about 10 wt % to about 40 wt % of the nitrogen-rich carbon material is nitrogen.

Embodiment 63 provides a nitrogen-rich carbon material made by the method of any one of Embodiments 61-62.

Embodiment 64 provides the method, composition, or material of any one or any combination of Embodiments 1-63 optionally configured such that all elements or options recited are available to use or select from.

What is claimed is:

1. A functionalized carbon matrix having the structure: 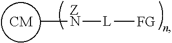
wherein
wherein at each occurrence Z is independently selected from H and a bond to CM,
CM is a substituted or unsubstituted carbon matrix,
at each occurrence linker L is independently substituted or unsubstituted (C₁-C₄₀)hydrocarbyl interrupted by 0, 1, 2, or 3 atoms chosen from —S—, —O—, and —NR³—,
at each occurrence functional group FG is independently chosen from —OR³, —OOR³, —OC(O)N(R³)₂, —CN, —CF₃, —OCF₃, —C(O), methylenedioxy, ethylenedioxy, —N(R³)₂, —SR³, —SOR³, —SO₂N(R³)₂, —C(O)R³, —C(O)C(O)R³, —C(O)CH₂C(O)R³, —C(S)R³, —C(O)OR³, —OC(O)R³, —C(O)N(R³)₂, —OC(O)N(R³)₂, —C(S)N(R³)₂, —N(R³)C(O)R³, —N(R³)N(R³)₂, —N(R³)N(R³)C(O)R³, —N(R³)N(R³)C(O)OR³, —N(R³)N(R³)CON(R³)₂, —N(R³)SO₂R³, —N(R³)SO₂N(R³)₂, —N(R³)C(O)OR³, —N(R³)C(O)R³, —N(R³)C(S)R³, —N(R³)C(O)N(R³)₂, —N(R³)C(S)N(R³)₂, —N(COR³)COR³, —N(OR³)R³, —C(═NH)N(R³)₂, —C(O)N(OR³)R³, —C(═NOR³)R³, —S(O)(OR¹)₃, —S(O)(OR¹)₂R², —S(O)(OR¹)R²₂, —S(O)R²₂, —OS(O)(OR¹)₃, —OS(O)(OR¹)₂R², —OS(O)(OR¹)R²₂, —OS(O)R²₂, —S(O)₂OR¹, —S(O)₂R², —OS(O)₂OR¹, —OS(O)₂R², —P(O)(OR¹)₂, —P(O)(OR¹)R², —P(O)R²₂, —OP(O)(OR¹)₂, —OP(O)(OR¹)R², —OP(O)R²₂, and a substituted or unsubstituted nitrogen-containing (C₁-C₂₀)heterocycle, wherein
at each occurrence R¹ is independently chosen from —H, a counterion, and substituted or unsubstituted (C₁-C₂₀)hydrocarbyl,
at each occurrence R² is independently chosen from substituted or unsubstituted (C₁-C₂₀)hydrocarbyl, and
at each occurrence R³ is independently chosen from —H and substituted or unsubstituted (C₁-C₂₀)hydrocarbyl, and
degree of substitution n is about 1 to about 1,000,000.

2. The functionalized carbon matrix of claim 1, wherein the functionalized carbon matrix is a reaction product of at least one of hydrothermal carbonization and pyrolysis of a reducing sugar and an amine having the structure H₂N-L-FG.

3. The functionalized carbon matrix of claim 1, wherein at each occurrence L is independently chosen from methylene, ethylene, propylene, butylene, pentylene, hexylene, heptylene, octylene, nonylene, decylene, and phenylene.

4. The functionalized carbon matrix of claim 1, wherein at each occurrence FG is independently chosen from an acidic functionality or a salt thereof.

5. The functionalized carbon matrix of claim 1, wherein at each occurrence FG is independently chosen from S(O)₂OR¹ and P(O)(OR¹)₂, wherein at each occurrence R¹ is independently selected from H, (C₁-C₅)alkyl, and a counterion.

6. The functionalized carbon matrix of claim 1, wherein at each occurrence FG is independently chosen from a basic functionality.

7. The functionalized carbon matrix of claim 1, wherein at each occurrence FG is independently chosen from substituted or unsubstituted pyridinyl and substituted or unsubstituted piperidinyl.

8. The functionalized carbon matrix of claim 1, wherein at each occurrence L is independently chosen from ethylene, butylene, methylene, and phenylene, and at each occurrence FG is independently chosen from S(O)₂OH, P(O)(OH)₂, pyridin-4-yl, and piperidin-4-yl.

9. A composition comprising the functionalized carbon material of claim 1.

10. A compound comprising:
the functionalized carbon matrix of claim 1; and
at least one metal cation coordinated thereto.

11. The compound of claim 10, wherein the metal cation is chosen from at least one of Cu⁺, Cu²⁺, Fe²⁺, Fe³⁺, Pb²⁺, Pb⁴⁺, Sn²⁺, Sn⁴⁺, Ni²⁺, Ni³⁺, Hg₂²⁺, Hg²⁺, Cr²⁺, Cr³⁺, Mn²⁺, Mn³⁺, Co²⁺, Co³⁺, Ag⁺, Zn²⁺, Cd²⁺, Sc³⁺, Na⁺, K⁺, Mg²⁺, Pd²⁺, Pt²⁺, Li⁺, Al³⁺, Cs⁺, V²⁺, V⁴⁺, Ru³⁺, Ru⁴⁺, Rh³⁺, Ce³⁺, and Ca²⁺.

12. The compound of claim 10, wherein about 10 wt % to about 80 wt % of the compound is the metal cation.

13. A nanomaterial comprising the functionalized carbon matrix of claim 1.

14. A composite material, comprising
a high surface area substrate;
the functionalized carbon matrix of claim 1 deposited thereon.

15. A functionalized carbon matrix having the structure: 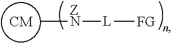
wherein
at each occurrence Z is independently selected from H and a bond to CM,
CM is a substituted or unsubstituted carbon matrix,
at each occurrence L is chosen from ethylene, butylene, methylene, and phenylene,
at each occurrence FG is chosen from S(O)₂OH, P(O)(OH)₂, pyridin-4-yl, and piperidin-4-yl, and
degree of substitution n is about 1 to about 1,000,000.

16. A method of making a functionalized carbon matrix, the method comprising
obtaining or providing a composition comprising
a reducing sugar, and
a functionalized amine having the structure H₂N-L-FG, wherein
at each occurrence linker L is independently substituted or unsubstituted (C₁-C₄₀)hydrocarbyl interrupted by 0, 1, 2, or 3 atoms chosen from —S—, —O—, and —NR³—,
at each occurrence functional group FG is independently chosen from —OR³, —OOR³, —OC(O)N(R³)₂, —CN, —CF₃, —OCF₃, —C(O), methylenedioxy, ethylenedioxy, —N(R³)₂, —SR³, —SOR³, —SO₂N(R³)₂, —C(O)R³, —C(O)C(O)R³, —C(O)CH₂C(O)R³, —C(S)R³, —C(O)OR³, —OC(O)R³, —C(O)N(R³)₂, —OC(O)N(R³)₂, —C(S)N(R³)₂, —N(R³)C(O)R³, —N(R³)N(R³)₂, —N(R³)N(R³)C(O)R³, —N(R³)N(R³)C(O)OR³, —N(R³)N(R³)CON(R³)₂, —N(R³)SO₂R³, —N(R³)SO₂N(R³)₂, —N(R³)C(O)OR³, —N(R³)C(O)R³, —N(R³)C(S)R³, —N(R³)C(O)N(R³)₂, —N(R³)C(S)N(R³)₂, —N(COR³)COR³, —N(OR³)R³, —C(═NH)N(R³)₂, —C(O)N(OR³)R³, —C(═NOR³)R³, —S(O)(OR¹)₃, —S(O)(OR¹)₂R², —S(O)(OR¹)R²₂, —S(O)R²₂, —OS(O)(OR¹)₃, —OS(O)(OR¹)₂R², —OS(O)(OR¹)R²₂, —OS(O)R²₂, —S(O)₂OR¹, —S(O)₂R², —OS(O)₂OR¹, —OS(O)₂R², —P(O)(OR¹)₂, —P(O)(OR¹)R², —P(O)R²₂, —OP(O)(OR¹)₂, —OP(O)(OR¹)R², —OP(O)R²₂, and a substituted or unsubstituted nitrogen-containing (C₁-C₂₀)heterocycle, wherein
at each occurrence R¹ is independently chosen from —H, a counterion, and substituted or unsubstituted (C₁-C₂₀)hydrocarbyl,
at each occurrence R² is independently chosen from substituted or unsubstituted (C₁-C₂₀)hydrocarbyl, and
at each occurrence R³ is independently chosen from —H and substituted or unsubstituted (C₁-C₂₀)hydrocarbyl; and
subjecting the composition to at least one of pyrolysis and hydrothermal carbonization, to provide a functionalized carbon matrix having the structure: 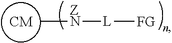
wherein at each occurrence Z is independently selected from H and a bond to CM, CM is a substituted or unsubstituted carbon matrix, and degree of substitution n is about 1 to about 1,000,000.

17. The method of claim 16, wherein the reducing sugar is at least one chosen from glucose, fructose, glyceraldehyde, galactose, lactose, maltose, erythrose, threose, ribose, arabinose, xylose, lyxose, allose, altrose, gulose, idose, talose, ribulose, xylulose, psicose, sorbose, tagatose, and cellobiose.

18. The method of claim 16, wherein the reducing sugar is glucose.

19. The method of claim 16, wherein the functionalized amine is chosen from aminoethyl sulfonic acid, aminoethyl phosphonic acid, 4-(2-aminoethyl)pyridine, and 4-aminomethylpiperidine.

20. A functionalized carbon material made by the method of claim 16.
